(19) United States
(12) Patent Application Publication
(10) Pub. No.: US 2008/0221060 A1
(43) Pub. Date: Sep. 11, 2008

(54) Therapeutic Compounds

(76) Inventors: Martyn Pritchard, Cambs (GB); Jacqueline Ouzman, Cambridge (GB); Edward Savory, Cambridge (GB); Giles Brown, Cambridge (GB)

Correspondence Address: FISH & RICHARDSON PC, P.O. BOX 1022, MINNEAPOLIS, MN, 55440-1022, US

(21) Appl. No.: 10/598,520

(22) PCT Filed: Mar. 4, 2005
(86) PCT No.: PCT/GB2005/000800

§ 371 Date: Dec. 7, 2007

(30) Foreign Application Priority Data
Mar. 5, 2004 (GB) 0405009.2
Mar. 5, 2004 (GB) 0405012.6
Mar. 5, 2004 (GB) PCT/GB04/00902
Jun. 2, 2004 (GB) 0412261.0
Jun. 2, 2004 (GB) 0412262.8
Jun. 18, 2004 (GB) 0413627.1
Sep. 6, 2004 (GB) 0419718.2
Sep. 9, 2004 (GB) 0420063.0
Sep. 16, 2004 (GB) 0420615.7

(51) Int. Cl. A61K 31/70; C07H 19/16; A61P 35/00

(52) U.S. Cl. 514/46; 536/27.6

(57) ABSTRACT

Use of compounds of general formula (A) as medicaments is described, in particular for the treatment of pain or inflammation; wherein: (I) when X=OH, R₂=NH₂, R₅=CH₂OH, R₆=H, R₁ is C₅-C₆ alkoxy, OCH₂Cyclopropyl, O-(2,2,3,3-tetrafluoro-cycloButyl), phenoxy, substituted phenoxy, OCH₂CH₂OH, or OCH₂CHF₂, (5-indanyl)oxy, C₁, C₂, C₅, or C₆ alkylamino, (R) or (S)-sec-Butylamino, C₅ or C₆ cycloalkylamino, exo-norbornane amino, (N-methyl, N-isoamylamino), phenylamino, phenylamino with either methoxy or fluoro substituents, a C₂ sulfone group, a C₂ alkyl group, a cyano group, a CONH₂ group, or 3,5-dimethylphenyl; or when X=H, R₂=NH₂, R₅=CH₂OH, R₆=H, R₁ is n-hexyloxy; or (II) when X=OH, R₁=H, R₅=CH₂OH, R₆=H, R₂ is NMe₂, N-(2-isopentenyl), piperazinyl, (N-Me, N-benzyl), (N-Me, N—CH₂Ph(3-Br)), (N-Me, N—CH₂Ph(3-CF₃)), or (N-Me, N-(2-methoxyethyl)), or OCH₂Cyclopentyl; or (III) when X=OH, R₅=CONHR₃, R₆=H: R₁ is H, R₃ is an isopropyl group, and R₂ is either NH₂ or a methylamino group (NHMe) or an isoamyl group (CH₂CH₂CHMe₂); or R₁ is H, R₃ is H, and R₂ is NH₂; or R₁ is OMe, R₃ is Ph, and R₂ is NH₂; or R₁ is NHCH₂CH₂CH₂CH₂CH₂Me, R₃ is CH₂CH₂CH₂Me, and R₂ is NH₂; or (IV) when X=OH, R₁=H, R₂=NH₂, R₅=CH₂NHCOR₄, R₆=H, R₁ is n-propyl or NHCH₂CH₃; or (V) when X=OH, R₅=CH₂OH, R₆=H: R₁ is NHCyclohexyl when R₂ is NMe₂; or R₁ is OMe when R₂ is NHBenzyl; or (VI) when X=OH, R₂=NH₂, R₅=CH₂OH, R₆=Me, R1 is NHCyclohexyl or NHCyclopentyl.

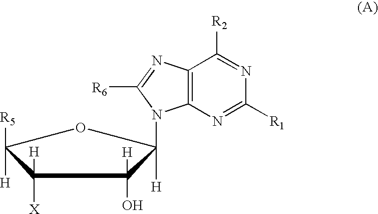

This invention relates to compounds that are adenosine receptor agonists, and to their use as therapeutic compounds, in particular as analgesic or anti-inflammatory compounds, or as disease-modifying antirheumatic drugs (DMARDs), and to methods of preventing, treating, or ameliorating pain or inflammation using these compounds.

Adenosine is a ubiquitous local hormone/neurotransmitter that acts on four known receptors, the adenosine A1, A2A, A2B and A3 receptors. Adenosine generally serves to balance the supply and demand of energy in tissues. For example, in the heart released adenosine slows the heart by an A1 receptor mediated action in the nodes and atria (Belardinelli, L & Isenberg, G Am. J. Physiol. 224, H734-H737), while simultaneously dilating the coronary artery to increase energy (i.e. glucose, fat and oxygen) supply (Knabb et al., Circ. Res. (1983) 53, 33-41). Similarly, during inflammation adenosine serves to inhibit inflammatory activity, while in conditions of excessive nerve activity (e.g. epilepsy) adenosine inhibits nerve firing (Klitgaard et al., Eur J. Pharmacol. (1993) 242, 221-228). This system, or a variant on it, is present in all tissues.

Adenosine itself can be used to diagnose and treat supraventricular tachycardia. Adenosine A1 receptor agonists are known to act as powerful analgesics (Sawynok, J. Eur J Pharmacol. (1998) 347, 1-11), and adenosine A2A receptor agonists are known to have anti-inflammatory activity (see, for example U.S. Pat. No. 5,877,180 and WO 99/34804). In experimental animals, A2A receptor agonists have been shown to be effective against a wide variety of conditions including sepsis, arthritis, and ischaemia/reperfusion injury arising from renal, coronary or cerebral artery occlusion. The common factor in these conditions is a reduction in the inflammatory response caused by the inhibitory effect of this receptor on most, if not all, inflammatory cells.

However, the ubiquitous distribution of adenosine receptors means that administration of adenosine receptor agonists causes adverse side effects. This has generally precluded the development of adenosine-based therapies. Selective A1 receptor agonists cause bradycardia. A2A receptor agonists cause widespread vasodilation with consequent hypotension and tachycardia. The first selective A2A receptor agonist (2-[4-(2-carboxyethyl)phenylethylamino]-5′-N-ethylcarboxamidoadenosine, or CGS21680), was tested in a Phase 2A clinical trial as a potential anti-hypertensive. However, administration of this compound caused a large fall in blood pressure and consequent increase in cardiac output. This has prevented use of CGS21680 as a medicament. Webb et al (J. Pharmacol Exp Ther (1991) 259, 1203-1212), Casati et al. (J Pharmacol Exp Ther (1995) 275(2):914-919), and Bonnizone et al. (Hypertension. (1995) 25, 564-9) show that selective A2A adenosine receptor agonists cause hypotension and tachycardia. The degree of tachycardia induced is sufficient to preclude their use as medicaments. Alberti et al. (J Cardiovasc Pharmacol. 1997 September; 30(3):320-4) discloses that selective A2A adenosine receptor agonists are potent vasodilators that reduce blood pressure and induce marked increments in heart rate and plasma renin activity. These side effects preclude their use as medicaments.

U.S. Pat. No. 5,877,180 relates to agonists of A2A adenosine receptors which are stated to be effective for the treatment of inflammatory diseases. The preferred agonists, WRC0090 and SHA 211 (WRC0474), are disclosed to be more potent and selective than previously reported adenosine analogs such as CGS21680 and CV1808. Administration of SHA 211 or WRC0090 is considered to reduce the possibility of side effects mediated by the binding of the analogs to other adenosine receptors. However, only in vitro data relating to the activity of SHA 211 is included. There is no demonstration that any of the compounds described could be therapeutically effective in vivo without causing serious side effects. Although side effects mediated by the binding of potent and selective adenosine A2A receptor agonists to other adenosine receptors is expected to be reduced by use of such agonists, the ubiquitous distribution of adenosine receptors means that these compounds would still be expected to activate adenosine A2A receptors in normal tissue and, therefore, cause serious side effects (such as hypotension and reflex tachycardia).

U.S. Pat. No. 3,936,439 discloses use of 2,6-diaminonebularine derivatives as coronary dilating and/or platelet aggregation inhibitory agents for mammals. In vivo data in dogs is included to support the coronary dilating action of N²-Phenyl-2,6-diaminonebularine, N²-Cyclohexyl-2,6-diaminonebularine, N²-(p-methoxyphenyl)-2,6-diaminonebularine, and N-Ethyl-2,6-diaminonebularine, and in vitro data supports the platelet aggregation inhibitory action of N²-Phenyl-2,6-diaminonebularine, N²-cyclohexyl-2,6-diaminonebularine, 2,6-Diaminonebularine, and N²-Ethyl-2,6-diaminonebularine. FR 2162128 (Takeda Chemical Industries, Ltd) discloses that adenosine derivatives (including 2-alkoxy adenosine derivatives comprising a lower alkyl group of not less than two carbon atoms) have hypotensive and coronary vasodilatory activity. In vivo data in dogs supports the coronary vasodilatory activity of 2-n-pentyloxyadenosine, 2-(β-hydroxyethoxy)-adenosine, and 2-phenoxyadenosine. However, there is no demonstration in U.S. Pat. No. 3,936,439 or FR 2162128 that any of the compounds described could be administered without causing serious side effects.

Ribeiro et al. (Progress in Neurobiology 68 (2003) 377-392) is a review of adenosine receptors in the nervous system. It is stated in the concluding remarks of this article (on page 387, right column, lines 4-10 of section 8) that “as noted a long time ago, activation of adenosine receptors at the periphery is associated with hypotension, bradycardia and hypothermia . . . . These side effects have so far significantly limited the clinical usefulness of adenosine receptor agonists”.

There is, therefore, a need to provide adenosine receptor agonists that can be administered with minimal side effects.

Certain aspects of the invention relate to the treatment of pain. Pain has two components, each involving activation of sensory neurons. The first component is the early or immediate phase when a sensory neuron is stimulated, for instance as the result of heat or pressure on the skin. The second component is the consequence of an increased sensitivity of the sensory mechanisms innervating tissue which has been previously damaged. This second component is referred to as hyperlagesia, and is involved in all forms of chronic pain arising from tissue damage, but not in the early or immediate phase of pain perception.

Thus, hyperalgesia is a condition of heightened pain perception caused by tissue damage. This condition is a natural response of the nervous system apparently designed to encourage protection of the damaged tissue by an injured individual, to give time for tissue repair to occur. There are two known underlying causes of this condition, an increase in sensory neuron activity, and a change in neuronal processing of nociceptive information which occurs in the spinal cord. Hyperalgesia can be debilitating in conditions of chronic inflammation (e.g. rheumatoid arthritis), and when sensory nerve damage has occurred (i.e. neuropathic pain).

Two major classes of analgesics are known: (i) non steroidal anti-inflammatory drugs (NSAIDs) and the related COX-2 inhibitors; and (ii) opiates based on morphine. Analgesics of both classes are effective in controlling normal, immediate or nociceptive pain. However, they are less effective against some types of hyperalgesic pain, such as neuropathic pain. Many medical practitioners are reluctant to prescribe opiates at the high doses required to affect neuropathic pain because of the side effects caused by administration of these compounds (such as restlessness, nausea, and vomiting), and the possibility that patients may become addicted to them. NSAIDs are much less potent than opiates, so even higher doses of these compounds are required. However, this is undesirable because these compounds cause irritation of the gastrointestinal tract.

There is also a need to provide analgesics, particularly anti-hyperalgesics, which are sufficiently potent to control pain perception in neuropathic and other hyperalgesic syndromes, and which do not have serious side effects or cause patients to become addicted to them.

Spongosine was first isolated from the tropical marine sponge, Cryptotethia crypta in 1945 (Bergmann and Feeney, J. Org. Chem. (1951) 16, 981, Ibid (1956) 21, 226), and was the first methoxypurine found in nature. It is also known as 2-methoxyadenosine, or 9H-purin-6-amine, 9-α-D-arabinofuranosyl-2-methoxy. The first biological activities of spongosine were described by Bartlett et al. (J. Med. Chem. (1981) 24, 947-954). Spongosine (and other compounds) was tested for its skeletal muscle-relaxant, hypothermic, cardiovascular and anti-inflammatory effects in rodents following oral administration (anti-inflammatory activity was assessed by inhibition of carageenan-induced oedema in a rat paw). Spongosine caused 25% inhibition of carageenan-induced inflammation in rats at 20 mg/kg po. However, reductions in mean blood pressure (41%), and in heart rate (25%) were also observed after administration of this compound at this dose.

The affinity of spongosine for the rat adenosine A1 and A2A receptors has been determined. The Kd values obtained (in the rat) were 340 nM for the A1 receptor and 1.4 μM for the A2A receptor, while the EC50 value for stimulation of the rat A2A receptor was shown to be 3 μM (Daly et al., Pharmacol. (1993) 46, 91-100). In the guinea pig, the efficacy of spongosine was tested in the isolated heart preparation and the EC50 values obtained were 10 μM and 0.7 μM for the adenosine A1 and A2A receptors, respectively (Ueeda et al J Med Chem (1991) 34, 1334-1339). Because of the low potency and poor receptor selectivity of this compound it was largely ignored in favour of more potent and receptor selective adenosine receptor agonists.

It has surprisingly been found that spongosine is an effective analgesic at doses as much as one hundred times lower than would be expected to be required based on the known affinity of this compound for adenosine receptors. At these doses, spongosine does not cause the significant side effects associated with higher doses of this compound, or other adenosine receptor agonists. Thus, the therapeutic effects of spongosine can be separated from its side effects. The activity of spongosine as an analgesic is the subject of International patent application no. PCT/GB03/05379, and the activity of compounds related to spongosine as analgesics is the subject of International patent application no. PCT/GB04/00935. Use of spongosine and related compounds to treat inflammation and other disorders is the subject of International patent application no. PCT/GB04/000952.

The Applicant has found that spongosine, and the related compounds described in PCT/GB04/00935 and PCT/GB04/000952, have increased affinity for adenosine receptors at pH below pH 7.4. It is believed that this property explains the surprising activity of these compounds at low doses. The Applicant has been able to identify certain other compounds that also have increased affinity for adenosine receptors at reduced pH. It is thought that these compounds can be used as medicaments without causing serious side effects.

According to the invention there are provided adenosine receptor agonists of the following formulae:

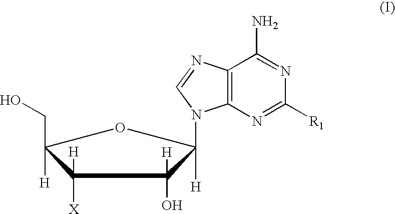

wherein:
when X=OH, R₁ is C₁ or C₄-C₆ alkoxy (preferably C₅-C₆ alkoxy), OCH₂Cyclopropyl, OCH₂Cyclopentyl, O-(2,2,3,3-tetrafluoro-cycloButyl), phenoxy, substituted phenoxy (preferably substituted with nitrile (preferably 4-nitrile), 4-methyl, phenyl (preferably 3-phenyl), 3-bromo, 3-isopropyl, 2-methyl, 2,4-difluoro, 2,5-difluoro, 3,4-difluoro, 2,3,5-trifluoro, or (3-methyl,4-fluoro)), OCH₂CH₂OH, OCH₂CHF₂, (5-indanyl)oxy, C₁, C₂, C₅, or C₆ alkylamino, (R) or (S)-sec-Butylamino, C₅ or C₆ cycloalkylamino, exo-norbornane amino, (N-methyl, N-isoamylamino), phenylamino, phenylamino with either methoxy or fluoro substituents, a C₂ sulfone group, a C₇ alkyl group, a cyano group, a CONH₂ group, or 3,5-dimethylphenyl; or when X=H, R₁ is n-hexyloxy;

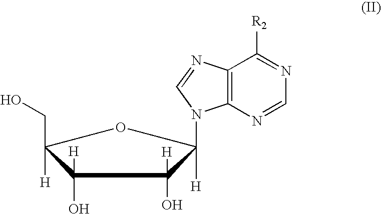

wherein R₂ is NMe₂, N-(2-isopentenyl), piperazinyl, (N-Me, N-benzyl), (N-Me, N—CH₂Ph(3-Br)), (N-Me, N—CH₂Ph(3-CF₃)), or (N-Me, N-(2-methoxyethyl)), or OCH₂Cyclopentyl;

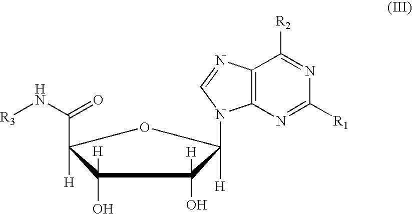

wherein:
when R₁=H, R₃ is an isopropyl group, and R₂ is either NH₂, a methylamino group (NHMe) or an isoamyl group (CH₂CH₂CHMe₂); or
when R₁=H, R₃ is H, and R₂ is NH₂; or
when R₁ is OMe, R₃ is Ph, and R₂ is NH₂; or
when R₁ is NHCH₂CH₂CH₂CH₂CH₂Me, R₃ is CH₂CH₂CH₂Me, and R₂ is NH₂;

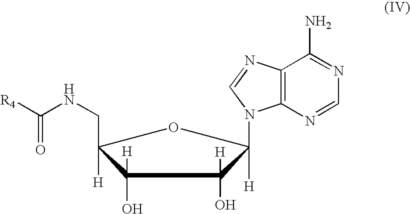

wherein R₄ is n-propyl or NHCH₂CH₃;

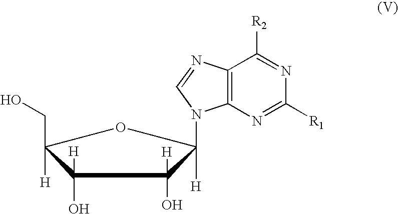

wherein:
R₁ is NHCyclohexyl when R₂ is NMe₂; or
R₁ is OMe when R₂ is NHBenzyl;

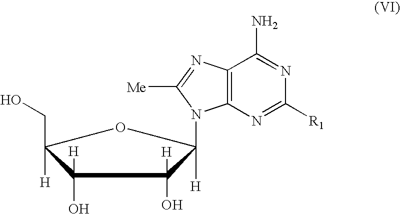

wherein R1 is NHCyclohexyl, NHCyclopentyl, or NH-n-Hexyl;
or a pharmaceutically acceptable salt thereof.

The term “alkyl” is used herein to mean an unsubstituted straight or branched chain hydrocarbon group. Preferably the alkyl is straight chain.

The term “alkoxy” is used herein to mean an unsubstituted straight or branched chain alkyl-oxy group. Preferably the alkoxy is a straight chain alkyl-oxy group.

The term “C₁, C₂, C₅, or C₆ alkylamino” is used herein to mean a group —NR^{x}R^{y} in which R^{x} is hydrogen and R^{y} is C₁, C₂, C₅, or C₆ alkyl, or in which R^{x} and R^{y} are each independently C₁, C₂, C₅, or C₆ alkyl. Preferably R^{x} and R^{y} are each C₁ alkyl.

Preferred compounds of formula (I) are compounds of formula (I)(a) or (I)(b):

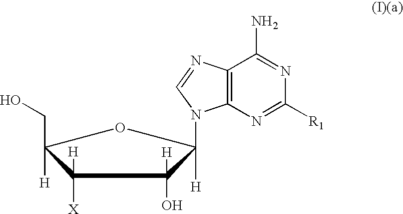

wherein:
when X=OH, R₁ is C₅-C₆ alkoxy, phenoxy substituted with nitrile (preferably 4-nitrile), phenyl (preferably 3-phenyl), or 3-isopropyl, (5-indanyl)oxy, C₅ or C₆ alkylamino, (N-methyl, N-isoamylamino), a C₂ sulfone group, or a C₇ alkyl group; or
when X=H, R₁ is n-hexyloxy;
or a pharmaceutically acceptable salt thereof;

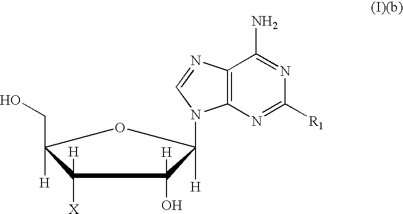

wherein:
when X=OH, R₁ is phenoxy, substituted phenoxy, C₁ or C₂ alkylamino, phenylamino with either methoxy or fluoro substituents, or OCH₂CH₂OH; or
when X=H, R₁ is n-hexyloxy; or a pharmaceutically acceptable salt thereof.

Preferred compounds of the invention are compound numbers 2, 3, 7-18, 22-25, 31-33, 35, 37, 40, 44, 45, 47, 48, or 51-61 as defined in Examples 1-6 below, or their pharmaceutically acceptable salts. Synthesis of these compounds is described in Examples 14-30 below.

There are also provided according to the invention methods of synthesis of compound numbers 2, 3, 7-18, 22-25, 31-33, 35, 37, 40, 44, 45, 47, 48, or 51-61 as set out in the claims below. In some cases the precursors of these compounds include one or more protecting groups. It will be appreciated that, if desired, other carboxy-based hydroxyl protecting groups may be used instead of those specified.

Compounds of the invention are all believed to have increased affinity for adenosine receptors at pH below pH 7.4. In normal mammalian tissues extracellular pH is tightly regulated between pH 7.35 and 7.45. Some tissues experience lower pH values, particularly the lumen of the stomach (pH between 2 and 3) and the surfaces of some epithelia (for example, the lung surface pH is approximately 6.8). In pathological tissues, for example during inflammation, ischaemia and other types of damage, a reduction in pH occurs.

Because of the increased affinity of compounds of the invention for adenosine receptors at reduced pH, it is thought that the actions of these compounds can be targeted to regions of low pH, such as pathological tissues. Consequently, the doses of these compounds that are required to give therapeutic effects are much lower than would be expected based on their affinity for adenosine receptors at normal extracellular physiological pH. Since only low doses of the compounds are required, the serious side effects associated with administration of adenosine receptor agonists are avoided or minimised. This has the surprising consequence (contrary to the teaching in the art, for example in U.S. Pat. No. 5,877,180) that some adenosine receptor agonists that are low affinity and/or non-selective agonists at physiological pH (such as spongosine) can be therapeutically effective without causing serious side effects.

Some compounds within the scope of formulae (I)-(VI) have previously been disclosed to be adenosine receptor agonists. However, it was not appreciated that the actions of these compounds could be targeted to pathological tissues or, therefore, that their therapeutic effects could be separated from their side effects. In view of the teaching of the present invention, it is believed that compounds of formulae (I)-(VI) can be administered at doses well below those expected to be required based on their affinity for adenosine receptors at pH 7.4, and cause therapeutic effects at these doses without causing serious side effects.

Thus, according to the invention there is provided a compound of the invention for use as a medicament.

It is believed that compounds of formulae (I)-(VI) have analgesic and/or anti-inflammatory activity and can be administered with reduced probability and severity of side effects compared to other adeno sine receptor agonists.

According to the invention there is provided use of a compound of formula (I), (II), (III), (IV), (V), or (VI) in the manufacture of a medicament for the prevention, treatment, or amelioration of pain, particularly hyperalgesia.

There is also provided according to the invention a method of preventing, treating, or ameliorating pain (particularly hyperalgesia) which comprises administering a compound of formula (I), (II), (III), (IV), (V), or (VI) to a subject in need of such prevention, treatment, or amelioration.

Preferred compounds of formula (I), (II), (III), (IV), (V), and (VI) are detailed in the Examples.

Compounds of formulae (I)-(VI) are believed to be effective in inhibiting pain perception in mammals suffering from pain, in particular neuropathic or inflammatory pain, even when administered at doses expected to give plasma concentrations well below those known to activate adenosine receptors. Therefore, it is believed that compounds of formulae (I)-(VI) can treat pain particularly neuropathic and inflammatory pain) without causing the significant side effects associated with administration of other adenosine receptor agonists.

As mentioned above hyperalgesia is a consequence in most instances of tissue damage, either damage directly to a sensory nerve, or damage of the tissue innervated by a given sensory nerve. Consequently, there are many conditions in which pain perception includes a component of hyperalgesia.

According to the invention there is provided use of a compound of formula (I), (II), (III), (IV), (V), or (VI) as an analgesic (particularly an anti-hyperalgesic) for the prevention, treatment, or amelioration of pain (particularly hyperalgesia) caused as a result of neuropathy, including Diabetic Neuropathy, Polyneuropathy, Cancer Pain, Fibromyalgia, Myofascial Pain Syndrome, Osteoarthritis, Pancreatic Pain, Pelvic/Perineal pain, Post Herpetic Neuralgia, Rheumatoid Arthritis, Sciatica/Lumbar Radiculopathy, Spinal Stenosis, Temporo-mandibular Joint Disorder, HIV pain, Trigeminal Neuralgia, Chronic Neuropathic Pain, Lower Back Pain, Failed Back Surgery pain, back pain, post-operative pain, post physical trauma pain (including gunshot, road traffic accident, burns), Cardiac pain, Chest pain, Pelvic pain/PID, Joint pain (tendonitis, bursitis, acute arthritis), Neck Pain, Bowel Pain, Phantom Limb Pain, Obstetric Pain (labour/C-Section), Renal Colic, Acute Herpes Zoster Pain, Acute Pancreatitis Breakthrough Pain (Cancer), Dysmenorhoea/Endometriosis.

According to the invention there is also provided use of a compound of formula (I), (II), (III), (IV), (V), or (VI) as an analgesic (particularly an anti-hyperalgesic) for the prevention, treatment, or amelioration of pain (particularly hyperalgesia) caused as a result of inflammatory disease, or as a result of combined inflammatory, autoimmune and neuropathic tissue damage, including rheumatoid arthritis, osteoarthritis, rheumatoid spondylitis, gouty arthritis, and other arthritic conditions, cancer, HIV, chronic pulmonary inflammatory disease, silicosis, pulmonary sarcosis, bone resorption diseases, reperfusion injury (including damage caused to organs as a consequence of reperfusion following ischaemic episodes e.g. myocardial infarcts, strokes), autoimmune damage (including multiple sclerosis, Guillam Barre Syndrome, myasthenia gravis) graft v. host rejection, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria and bacterial meningitis, bowel pain, cancer pain, back pain, fibromyalgia, post-operative pain.

It has also been appreciated that spongosine may be effective in the prevention, treatment, or amelioration of ischaemic pain. It is believed that compounds related to spongosine may also be effective against ischaemic pain.

According to the invention there is provided use of a compound of formula (VII) in the manufacture of a medicament for the prevention, treatment, or amelioration of pain, in particular ischaemic pain:

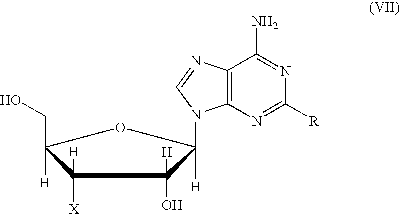

wherein R is C₁₋₄ alkoxy, and X is H or OH,
or a pharmaceutically acceptable salt thereof.

Preferably R is C₁₋₄ alkoxy, and X is OH, or a pharmaceutically acceptable salt thereof. Compounds of formula (VII) may exclude 2-methoxyadenosine (spongosine). There is also provided according to the invention a method of preventing, treating, or ameliorating pain, in particular ischaemic pain, which comprises administering a compound of formula (VII) to a subject in need of such prevention, treatment, or amelioration.

It has also been appreciated that compounds of formula (I)-(VI) may be effective in the prevention, treatment, or amelioration of ischaemic pain.

The term “ischaemic pain” is used herein to mean pain associated with a reduction in blood supply to a part of the body. A reduced blood supply limits the supply of oxygen (hypoxia) and energy to that part of the body. Ischaemia arises from poor blood perfusion of tissues and so ischaemic pain arises in coronary artery disease, peripheral artery disease, and conditions which are characterized by insufficient blood flow, usually secondary to atherosclerosis. Other vascular disorders can also result in ischaemic pain. These include: left ventricular hypertrophy, coronary artery disease, essential hypertension, acute hypertensive emergency, cardiomyopathy, heart insufficiency, exercise tolerance, chronic heart failure, arrhythmia, cardiac dysrhythmia, syncopy, arteriosclerosis, mild chronic heart failure, angina pectoris, Prinzmetal's (variant) angina, stable angina, and exercise induced angina, cardiac bypass reocclusion, intermittent claudication (arteriosclerosis oblitterens), arteritis, diastolic dysfunction and systolic dysfunction, atherosclerosis, post ischaemia/reperfusion injury, diabetes (both Types I and II), thromboembolisms. Haemorrhagic accidents can also result in ischaemic pain. In addition poor perfusion can result in neuropathic and inflammatory pain arising from hypoxia-induced nerve cell damage (e.g. in cardiac arrest or bypass operation, diabetes or neonatal distress).

Compounds of formulae (I)-(VII) are believed to be effective in prevention, treatment, or amelioration of ischaemic pain even when administered at doses expected to give plasma concentrations well below those known to activate adenosine receptors. At these doses, it is believed that the compounds do not cause the significant side effects associated with administration of higher doses of spongosine, or other adenosine receptor agonists.

There is further provided according to the invention use of a compound of the invention (i.e. a compound of formula (I), (II), (III), (IV), (V), (VI), or (VII)) for the manufacture of a medicament for the prevention, treatment, or amelioration of inflammation.

There is further provided according to the invention a method of prevention, treatment, or amelioration of inflammation, which comprises administering a compound of the invention to a subject in need of such prevention, treatment, or amelioration.

In particular, it is believed that compounds of the invention (i.e. compounds of formula (I), (II), (III), (IV), (V), (VI), or (VII)) can be used to prevent, treat, or ameliorate inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); autoimmune damage (including multiple sclerosis, Guillam Barre Syndrome, myasthenia gravis); obesity; cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), muscle fatigue or muscle cramp (particularly athletes' cramp), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), cerebral malaria, bacterial meningitis, TNF-enhanced HIV replication, TNF inhibition of AZT and DDI activity, osteoporosis and other bone resorption diseases, osteoarthritis, rheumatoid arthritis, infertility from endometriosis, fever and myalgia due to infection, cachexia secondary to cancer, cachexia secondary to infection or malignancy, cachexia secondary to acquired immune deficiency syndrome (AIDS), AIDS related complex (ARC), keloid formation, scar tissue formation, adverse effects from amphotericin B treatment, adverse effects from interleukin-2 treatment, adverse effects from OKT3 treatment, or adverse effects from GM-CSF treatment, and other conditions mediated by excessive anti-inflammatory cell (including neutrophil, eosinophil, macrophage and T-cell) activity.

Continuous low grade inflammation is known to be associated with obesity (in the presence and absence of insulin resistance and Type II diabetes) (Browning et al (2004) Metabolism 53, 899-903, Inflammatory markers elevated in blood of obese women; Mangge et al (2004) Exp Clin Endocrinol Diabetes 112, 378-382, Juvenile obesity correlates with serum inflammatory marker C-reactive protein; Maachi et al Int J Obes Relat Metab Disord. 2004 28, 993-997, Systemic low grade inflammation in obese people). A possible reason for this is that fat cells secrete TNF alpha and interleukins 1 and 6, which are pro-inflammatory.

Compounds of the invention that are selective agonists of adenosine A2A and/or A3 receptors are particularly preferred because it is believed that such compounds will have strong anti-inflammatory activity. By selective agonists of adenosine A2A and/or A3 receptors is meant agonists that activate adenosine A2A and/or A3 receptors at concentrations that are lower (preferably one thousandth to one fifth) than required to activate adenosine A1 receptors. Furthermore, A1 receptors have pro-inflammatory activity, so such effects are expected to be minimised for compounds that are selective for A2A and/or A3 receptors.

It will be appreciated that any pathological condition that can be prevented or improved by agonism of adenosine A2A and/or A3 receptors can be prevented, treated, or ameliorated by compounds of formulae (I)-(VII).

According to the invention there is provided use of a compound of formula (I)-(VII) in the manufacture of a medicament for the prevention, treatment, or amelioration of a pathological condition that can be improved or prevented by agonism of adenosine A2A and/or A3 receptors.

There is also provided according to the invention a method of prevention, treatment, or amelioration of a pathological condition that can be improved or prevented by agonism of adenosine A2A and/or A3 receptors, which comprises administering a compound of formula (I)-(VII) to a subject in need of such prevention, treatment, or amelioration.

A person of ordinary skill in the art can readily test whether or not a pathological condition that is prevented, treated, or ameliorated by a compound of formula (I)-(VII) is acting via adenosine A2A and/or A3 receptors. For example, this may be done by comparing the effect of the compound in an animal model of the pathological condition in the presence and absence of a selective antagonist of an adenosine A2A and/or A3 receptor. If the effect of the compound in the presence of the antagonist is reduced or absent compared with the effect of the compound in the absence of the antagonist, it is concluded that the compound is exerting its effect via an adenosine A2A and/or A3 receptor. Antagonists of adenosine A2A and A3 receptors are known to those of ordinary skill in the art (see for example Ongini et al., Farmaco. 2001 January-February; 56(1-2):87-90; Muller, Curr Top Med Chem. 2003; 3(4):445-62).

Alternatively, an adenosine A2A receptor knockout mouse may be used (Ohta A and Sitkovsky M, Nature 2001; 414:916-20). For example, the effect of the compound on a mouse that has symptoms of the pathological condition is compared with its effect on an adenosine A2A knockout mouse that has corresponding symptoms. If the compound is only effective in the mouse that has adenosine A2A receptors it is concluded that the compound is exerting its effect via adenosine A2A receptors.

Compounds of the invention (i.e. compounds of formula (I), (II), (III), (IV), (V), (VI), or (VII)) are believed to be much more effective at low doses than other adenosine receptor agonists. Thus, it is expected that compounds of the invention can be effectively administered at doses at which they have reduced probability and severity of side effects, or at which side effects are not observed. Such compounds provide significant advantages over the vast majority of other adenosine receptor agonists which only have anti-inflammatory effects at the same concentrations at which serious side effects are observed.

Compounds of the invention may alternatively or additionally have reduced probability and severity of side effects compared to other adenosine receptor agonists.

It is also believed that compounds of the invention (i.e. compounds of formula (I), (II), (III), (IV), (V), (VI), or (VII)) may be effective as disease-modifying anti-rheumatic drugs (DMARDs), in particular for use in the prevention, treatment, or amelioration of rheumatoid arthritis, and possibly other arthropathies such as osteoarthritis.

Medications used to treat rheumatoid arthritis (RA) can be divided into two groups: those that help relieve RA symptoms; and those that help modify the disease. Drugs that help to relieve RA symptoms include nonsteroidal anti-inflammatory drugs (NSAIDs) that relieve pain and reduce inflammation in the affected joints, analgesics (such as acetaminophen and narcotic pain medications) that relieve pain but do not slow joint damage or reduce inflammation, and corticosteroids that are anti-inflammatory drugs.

DMARDs help to improve RA symptoms (such as joint swelling and tenderness), but also slow the progression of joint damage caused by RA. Thus, while there is no cure for RA, DMARDs help to slow the progression of RA. In the past DMARDs were usually used to treat RA after NSAID therapy failed. However, DMARDs are now beginning to be used earlier in the course of RA because studies have suggested that early intervention with DMARDs offers important benefits. DMARDs and NSAIDs are often used in combination with each other.

Results from clinical studies have shown that known DMARDs slow the progression of RA. After 6 months of treatment, the rate of bone and cartilage damage had already started to slow in patients' joints. After 1 year, patients showed very little progression of joint damage, and after 2 years X rays showed that few patients in the study had newly damaged joints during the second year of treatment.

Examples of known DMARDs include sulphasalazine, penicillamine, chloroquine, hydroxychloroquine, gold (by intramuscular injection or orally as auranofin), methotrexate, cyclosporin, azathioprine, cyclophosphamide, leflunomide. More recently biological DMARDs have been developed which inhibit tumour necrosis factor alpha (TNF alpha). One example is Humira® which is indicated for reducing signs and symptoms and inhibiting the progression of structural damage in adults with moderately to severely active RA who have had an inadequate response to one or more DMARDs. Humira® is an anti-TNF alpha antibody.

Many of the known DMARDs cause serious side effects. Consequently, it is desired to provide new DMARDs that can be administered with minimal side effects.

Example 13 below shows the ability of spongosine to reduce phorbol ester induced TNF alpha release in U937 human macrophage cells. On this basis, it is believed that spongosine and related compounds of formula (I), (II), (III), (IV), (V), (VI), or (VII) also have DMARD activity.

According to the invention there is provided use of a compound of formula (I), (II), (III), (IV), (V), (VI), or (VII) in the manufacture of a medicament for slowing the progression of arthropathy.

There is also provided according to the invention a method of slowing the progression of arthropathy, which comprises administering a compound of formula (I), (II), (III), (IV), (V), (VI), or (VII) to a subject in need thereof.

Preferably the progression of RA is slowed, and in particular the progression of joint damage caused by RA.

A compound of the invention may be administered to the subject at any stage in the course of RA. A compound of the invention may be administered in combination with one or more NSAIDs or other DMARDs.

Compounds of the invention are believed to be effective as DMARDs even when administered at doses expected to give plasma concentrations well below those known to activate adenosine receptors. At these doses, it is believed that the compounds do not cause the significant side effects associated with administration of higher doses of spongosine, or other adenosine receptor agonists.

A particular advantage of use of compounds of the invention as DMARDs is that it is believed that they will be orally active, in contrast to anti-TNF alpha antibodies which must be injected.

It has also been appreciated that compounds of formulae (I)-(VII) may be effective in preventing, treating, or ameliorating macro and micro vascular complications of type 1 or 2 diabetes (including retinopathy, nephropathy, autonomic neuropathy), or blood vessel damage caused by ischaemia (either diabetic or otherwise) or atherosclerosis (either diabetic or otherwise).

According to the invention, there is provided use of a compound of formula (I), (II), (III), (IV), (V), (VI), or (VII) in the manufacture of a medicament for the prevention, treatment, or amelioration of macro or micro vascular complications of type 1 or 2 diabetes, retinopathy, nephropathy, autonomic neuropathy, or blood vessel damage caused by ischaemia or atherosclerosis.

According to the invention there is also provided a method of preventing, treating, or ameliorating macro or micro vascular complications of type 1 or 2 diabetes, retinopathy, nephropathy, autonomic neuropathy, or blood vessel damage caused by ischaemia or atherosclerosis, in a subject in need of such prevention, treatment, or amelioration, which comprises administering a compound of formula (I), (II), (III), (IV), (V), (VI), or (VII) to the subject.

Preferred compounds of formula (VII) are 2-methoxyadenosine (i.e. spongosine), 2-ethoxyadenosine, and 2-butyloxyadenosine.

Compounds of formulae (I)-(VII) are believed to be effective in prevention, treatment, or amelioration of macro or micro vascular complications of type 1 and 2 diabetes, including retinopathy, nephropathy, autonomic neuropathy, or blood vessel damage caused by ischaemia or atherosclerosis (either diabetic or otherwise)) even when administered at doses expected to give plasma concentrations well below those known to activate adenosine receptors. At these doses, it is believed that the compounds do not cause the significant side effects associated with administration of higher doses of spongosine, or other adenosine receptor agonists.

Compounds of formula (I)-(VII) are also believed to be effective in the promotion of wound healing. According to the invention there is provided use of a compound of formula (I), (II), (III), (IV), (V), (VI), or (VII) in the manufacture of a medicament for the promotion of wound healing. There is also provided according to the invention a method of promoting wound healing in a subject, which comprises administering a compound of formula (I), (II), (III), (IV), (V), (VI), or (VII) to the subject.

The amount of a compound of formula (I)-(VII) that is administered to a subject is preferably an amount which gives rise to a peak plasma concentration that is less than the EC50 value of the compound at adenosine receptors (preferably at pH 7.4).

It will be appreciated that the EC50 value of the compound is likely to be different for different adenosine receptors (i.e. the A1, A2A, A2B, A3 adenosine receptors). The amount of the compound that is to be administered should be calculated relative to the lowest EC50 value of the compound at the different receptors.

Thus, preferably the amount of a compound of the invention that is administered to a subject should be an amount which gives rise to a peak plasma concentration that is less than the lowest EC50 value of the compound at adenosine receptors.

Preferably the peak plasma concentration of the compound is one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one third, or one fiftieth to one half, or one fiftieth to one fifth, or one tenth to one half, or one tenth to one fifth) of the lowest EC50 value.

Preferably the amount of a compound of the invention that is administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the lowest EC50 value of the compound at adenosine receptors.

Preferably the amount administered gives rise to a plasma concentration that is maintained for more than one hour between one thousandth and one half, or one thousandth and one fifth, or one thousandth and one twentieth, or one hundredth and one half, or one hundredth and one fifth, or one fiftieth and one half, or one fiftieth and one fifth, of the EC50 value of the compound at adenosine receptors at pH 7.4.

For the avoidance of doubt, the EC50 value of a compound is defined herein as the concentration of the compound that provokes a receptor response halfway between the baseline receptor response and the maximum receptor response (as determined, for example, using a dose-response curve).

The EC50 value should be determined under standard conditions (balanced salt solutions buffered to pH 7.4). For EC50 determinations using isolated membranes, cells and tissues this would be in buffered salt solution at pH 7.4 (e.g. cell culture medium), for example as in Daly et al, Pharmacol. (1993) 46, 91-100), or preferably as in Tilburg et al (J. Med. Chem. (2002) 45, 91-100). The EC50 could also be determined in vivo by measuring adenosine receptor mediated responses in a normal healthy animal, or even in a tissue perfused under normal conditions (i.e. oxygenated blood, or oxygenated isotonic media, also buffered at pH 7.4) in a normal healthy animal.

Alternatively, the amount of a compound of the invention that is administered may be an amount that results in a peak plasma concentration that is less than the lowest or highest Kd value of the compound at adenosine receptors (i.e. less than the lowest or highest Kd value of the compound at A1, A2A, A2B, and A3 adenosine receptors). Preferably the peak plasma concentration of the compound is one ten thousandth to one half (or one ten thousandth to one fifth, or one ten thousandth to one twentieth, or one ten thousandth to one hundredth, or one ten thousandth to one thousandth, or one thousandth to one half, or one thousandth to one third, or one thousandth to one fifth, or one thousandth to one, twentieth, or one fiftieth to one tenth, or one hundredth to one half, or one hundredth to one fifth, or one fiftieth to one half, or one fiftieth to one fifth, or one tenth to one half, or one tenth to one fifth) of the lowest or highest Kd value.

Preferably the amount of the compound that is administered is an amount that results in a plasma concentration that is maintained for at least one hour between one thousandth and one half, or one thousandth and one fifth, more preferably between one thousandth and one twentieth, or one hundredth and one half, or one hundredth and one fifth, or one fiftieth and one half, or one fiftieth and one fifth, of the Kd value of the compound at adenosine receptors.

Preferably the amount of the compound that is administered is an amount that results in a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one fiftieth to one third, or one tenth to one half, or one tenth to one fifth) of the lowest or highest Kd value of the compound at adenosine receptors.

The Kd value of the compound at each receptor should be determined under standard conditions using plasma membranes as a source of the adenosine receptors derived either from tissues or cells endogenously expressing these receptors or from cells transfected with DNA vectors encoding the adenosine receptor genes. Alternatively whole cell preparations using cells expressing adenosine receptors can be used. Labelled ligands (e.g. radiolabelled) selective for the different receptors should be used in buffered (pH 7.4) salt solutions (see e.g. Tilburg et al, J. Med. Chem. (2002) 45, 420-429) to determine the binding affinity and thus the Kd of the compound at each receptor.

Alternatively, the amount of a compound of the invention that is administered may be an amount that is one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the minimum amount (or dose) of the compound that gives rise to bradycardia, hypotension or tachycardia side effects in animals of the same species as the subject to which the compound is to be administered. Preferably the amount administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the minimum amount of the compound that gives rise to the side effects.

Preferably the amount administered gives rise to a plasma concentration that is maintained for more than 1 hour between one thousandth and one half, or one thousandth and one twentieth, or one hundredth or one fiftieth and one half, or one hundredth or one fiftieth and one fifth of the minimum dose that gives rise to the side effects.

Alternatively, the amount of a compound of the invention that is administered may be an amount that gives rise to plasma concentrations that are one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the minimum plasma concentration of the compound that cause bradycardia, hypotension or tachycardia side effects in animals of the same species as the subject to which the compound is to be administered. Preferably the amount administered gives rise to a plasma concentration that is maintained for more than one hour at one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one fifth, or one tenth to one half, or one tenth to one fifth) of the minimum plasma concentration of the compound that causes the side effects.

Preferably the amount administered gives rise to a plasma concentration that is maintained for more than 1 hour between one thousandth and one half, or one thousandth and one twentieth, or one hundredth or one fiftieth and one half, or one hundredth or one fiftieth and one fifth, of the minimum plasma concentration that causes the side effects.

The appropriate dosage of a compound of the invention will vary with the age, sex, weight, and condition of the subject being treated, the potency of the compound (such as its EC50 value for an adenosine receptor), its half life, its absorption by the body, and the route of administration, etc. However, the appropriate dosage can readily be determined by one skilled in the art.

A suitable way to determine the appropriate dosage is to assess cardiovascular changes (for example by ecg and blood pressure monitoring) at or around the EC50 value of the compound for an adenosine receptor (preferably the receptor for which it has highest affinity) to determine the maximum tolerated dose. The therapeutically effective dose is then expected to be one ten thousandth to one half (or one ten thousandth to one fifth, or one ten thousandth to one twentieth, or one ten thousandth to one hundredth, or one ten thousandth to one thousandth, or one thousandth to one half, or one thousandth to one fifth, or one thousandth to one twentieth, or one fiftieth to one tenth, or one hundredth to one half, or one hundredth to one fifth, or one fiftieth to one half, or one fiftieth to one third, or one fiftieth to one fifth, or one tenth to one half, or one tenth to one fifth) of the maximum tolerated dose.

Example 31 below shows that for spongosine the dose should be less than 28 mg in humans. This dose gives rise to plasma concentrations between 0.5 and 0.9 μM (close to the Kd at adenosine A2A receptors at pH 7.4 see below). Based on this result, the preferred dosage range for spongosine is 0.03 to 0.3 mg/kg.

The minimum plasma concentration of spongosine giving maximal analgesic relief in a rat adjuvant model of arthritis was 0.06 μM, considerably less than the EC50 of spongosine at the adenosine A2A receptor which is approximately 1 μM. The preferred dosing levels in humans give maximum plasma concentrations between 0.005 and 0.5 μM which are significantly lower than those expected to give an analgesic or an anti-inflammatory effect by an action on this receptor.

Alternatively, appropriate therapeutic concentrations of compounds of the invention are expected to be approximately 10-20 times the Ki for an adenosine receptor (the receptor for which the compound has the highest affinity) at pH 5.5. Thus, for spongosine 15 to 30 nM is required whereas using the Ki at pH7.4 the concentration that is expected to be required is 20 to 30 μM.

It is expected that the amount of a compound of the invention that is administered should be 0.001-15 mg/kg. The amount may be less than 6 mg/kg. The amount may be at least 0.001, 0.01, 0.1, or 0.2 mg/kg. The amount may be less than 0.1, or 0.01 mg/kg. Preferred ranges are 0.001-10, 0.001-5, 0.001-2, 0.001-1, 0.001-0.1, 0.001-0.01, 0.01-15, 0.01-10, 0.01-5, 0.01-2, 0.01-1, 0.1-10, 0.1-5, 0.1-2, 0.1-1, 0.1-0.5, 0.1-0.4, 0.2-15, 0.2-10, 0.2-5, 0.2-2, 0.2-1.2, 0.2-1, 0.6-1.2, mg/kg.

Preferred doses for a human subject (for example a 70 kg subject) are less than 420 mg, preferably less than 28 mg, more preferably less than 21 mg, and preferably at least 0.07, 0.1, 0.7, or 0.8 mg, more preferably at least 3.5 or 7 mg. More preferably 7-70 mg, 14-70 mg, or 3.5-21 mg.

It is believed that the dosage amounts specified above are significantly lower (up to approximately 1000 times lower) than would be expected to be required for an analgesic or an anti-inflammatory effect based on the EC50 value of the compound at the adenosine A2A receptor.

The preferred dosage amounts specified above are aimed at producing plasma concentrations that are approximately one hundredth to one half of the EC50 value of the compound at the adenosine receptor for which it has highest affinity.

A compound of the invention may be administered with or without other therapeutic agents, for example analgesics or anti-inflammatories (such as opiates, steroids, NSAIDs, cannabinoids, tachykinin modulators, or bradykinin modulators) or anti-hyperalgesics (such as gabapentin, pregabalin, cannabinoids, sodium or calcium channel modulators, anti-epileptics or anti-depressants), or DMARDs.

In general, a compound of the invention may be administered by known means, in any suitable formulation, by any suitable route. A compound of the invention is preferably administered orally, parenterally, sublingually, transdermally, intrathecally, or transmucosally. Other suitable routes include intravenous, intramuscular, subcutaneous, inhaled, and topical. The amount of drug administered will typically be higher when administered orally than when administered, say, intravenously.

It will be appreciated that a compound of the invention may be administered together with a physiologically acceptable carrier, excipient, or diluent.

To maintain therapeutically effective plasma concentrations for extended periods of time, compounds of the invention may be incorporated into slow release formulations.

Suitable compositions, for example for oral administration, include solid unit dose forms, and those containing liquid, e.g. for injection, such as tablets, capsules, vials and ampoules, in which the active agent is formulated, by known means, with a physiologically acceptable excipient, diluent or carrier. Suitable diluents and carriers are known, and include, for example, lactose and talc, together with appropriate binding agents etc.

A unit dosage of a compound of the invention (i.e. a compound of formula (I), (II), (III), IV), (V), (VI), or (VII)) typically comprises up to 500 mg (for example 1 to 500 mg, or preferably) 5 to 500 mg) of the active agent. Preferably the active agent is in the form of a pharmaceutical composition comprising the active agent and a physiologically acceptable carrier, excipient, or diluent. Preferred dosage ranges (i.e. preferred amounts of the active ingredient in a unit dose) are 0.001-10, 0.001-5, 0.001-2, 0.001-1, 0.001-0.1, 0.001-0.01, 0.01-15, 0.01-10, 0.01-5, 0.01-2, 0.01-1, 0.1-10, 0.1-5, 0.1-2, 0.1-1, 0.1-0.5, 0.1-0.4, 0.2-15, 0.2-10, 0.2-5, 0.2-2, 0.2-1.2, 0.2-1, 0.5 to 1, 0.6-1.2, typically about 0.2 or 0.6, mg of the active agent per kg of the (human) subject. Preferred amounts of the active agent are less than 420 mg, preferably less than 28 mg, more preferably less than 21 mg, and preferably at least 0.07, 0.1, 0.7 or 0.5 mg, more preferably at least 3.5 or 7 mg. More preferably 7 to 70 mg, or 14 to 70 mg, 3.5 to 21 mg, 0.07-0.7 mg, or 0.7-7 mg. At these levels, it is believed that effective treatment can be achieved substantially without a concomitant fall (for example, no more than 10%) in blood pressure and/or increase in compensatory heart rate.

A unit dosage of a compound of the invention may further comprise one or more other therapeutic agents, for example analgesics, anti-inflammatories, anti-hyperalgesics, or DMARDs.

Preferably a compound of the invention is administered at a frequency of 2 or 3 times per day.

Compounds of the invention can also serve as a basis for identifying more effective drugs, or drugs that have further reduced side effects.

Examples of pharmaceutically acceptable salts are organic addition salts formed with acids which form a physiologically acceptable anion, for example, tosylate, methanesulphonate, malate, acetate, citrate, malonate, tartarate, succinate, benzoate, ascorbate, α-ketoglutarate, and α-glycerophosphate. Suitable inorganic salts may also be formed, including hydrochloride, sulphate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example calcium) salts of carboxylic acids can also be made.

Use of a compound of formula (VII) in the manufacture of a medicament for the prevention, treatment, or amelioration of ischaemic pain, or a method of prevention, treatment, or amelioration of ischaemic pain by administering a compound of formula (I) in accordance with the invention may exclude prevention, treatment, or amelioration of pain resulting from damage caused to organs as a consequence of reperfusion following an ischaemic episode, for example a myocardial infarct, or a stroke.

Use of a compound of formula (VII) in the manufacture of a medicament for the prevention, treatment, or amelioration of ischaemic pain in accordance with the invention may exclude use of 2-propoxyadenosine, 2-isopropoxyadenosine, 3′ deoxy 2 methoxyadenosine or 3′ deoxy 2 ethoxyadenosine.

A method of prevention, treatment, or amelioration of ischaemic pain by administering a compound of formula (VII) in accordance with the invention may exclude use of 2-propoxyadenosine, 2-isopropoxyadenosine, 3′ deoxy 2 methoxyadenosine or 3′ deoxy 2 ethoxyadenosine.

Compounds of formula (I) may exclude 2-phenylamino adenosine (compound number 26), 2-ethylamino adenosine (compound number 20), 2-cyclohexylamino adenosine (compound number 30), 2-(4-methoxy phenylamino) adenosine (compound number 27) 2-phenoxyadenosine (compound number 6), or 2-(β-hydroxyethoxy)-adenosine (compound number 34).

Embodiments of the invention are described in the following examples with reference to the accompanying drawings in which:

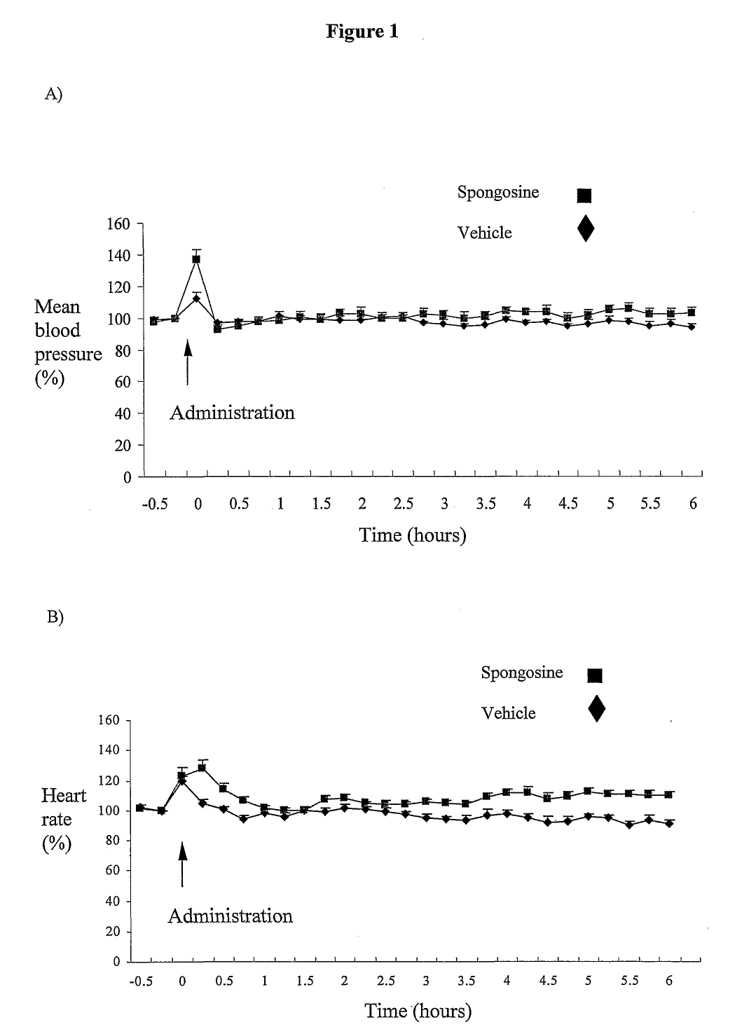

FIG. 1 shows the effect of spongosine (0.6 mg/kg p.o.) on A: blood pressure in normal rats; B: heart rate;

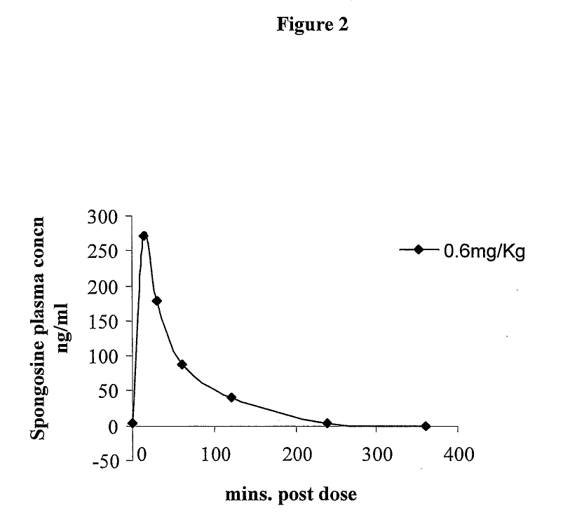

FIG. 2 shows the change in plasma concentration over time after administration of spongosine;

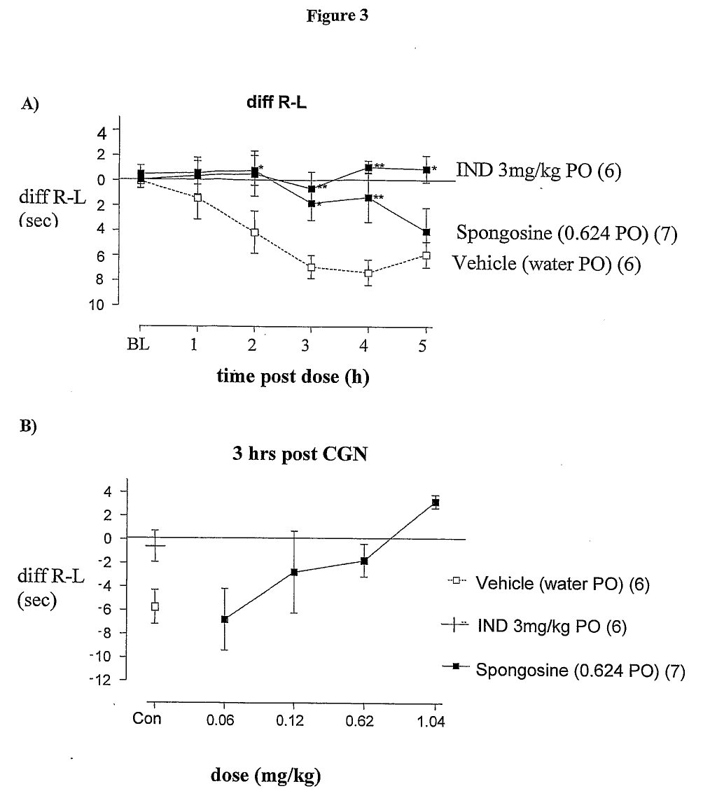

FIG. 3 shows the anti-hyperalgesic actions of spongosine (0.6 mg/kg p.o.) on carrageenan induced hyperalgesia. A: time course (*p<0.05, **p<0.01 versus vehicle (Sidak's), p>0.05 versus BL over 5 hrs for Spongosine and IND (Dunnett's)); B: dose dependency of the anti-hyperalgesic effect;

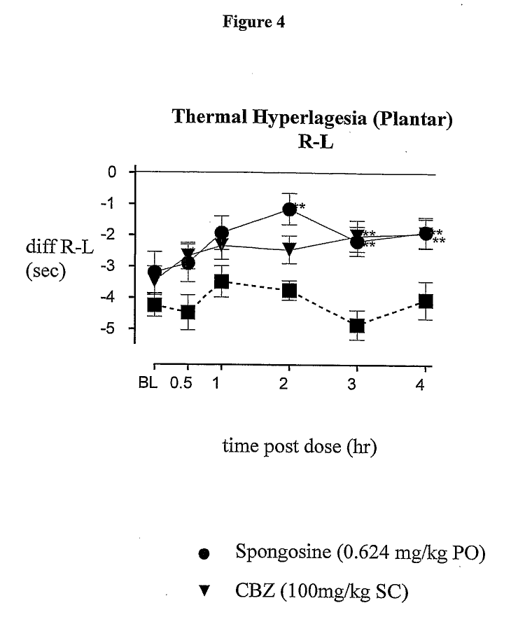

FIG. 4 shows the anti-hyperalgesic actions of spongosine (0.6 mg/kg p.o.) in the chronic constriction injury model of neuropathic pain (*p<0.05, **p<0.01 vs veh (ANOVA Sidak's);

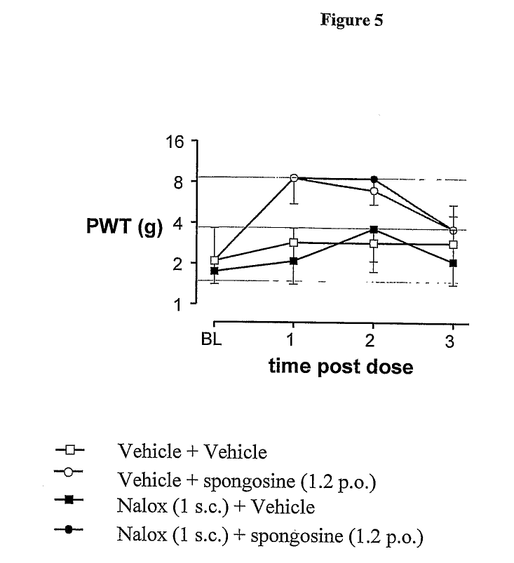

FIG. 5 shows the effect of spongosine (0.6 mg/kg p.o.) in the presence and absence of naloxone in the chronic constriction injury model of neuropathic pain;

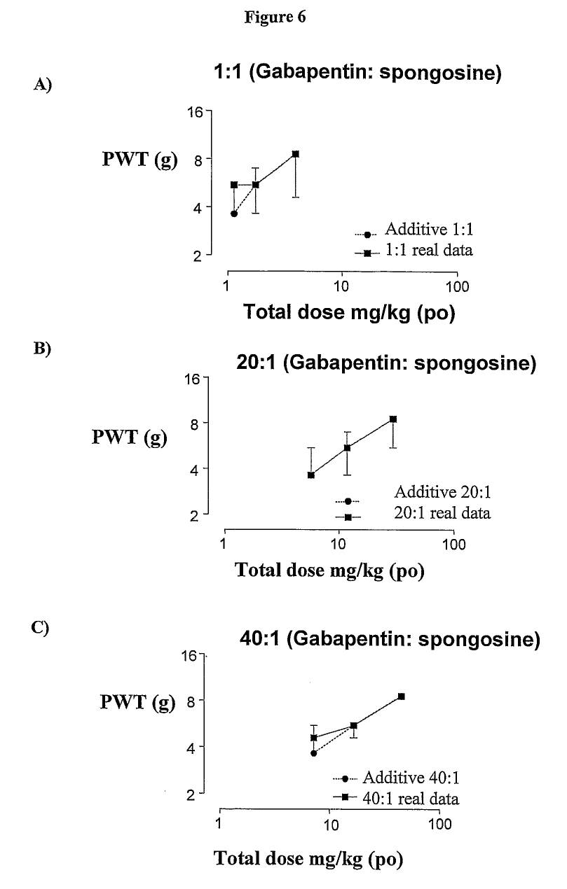

FIG. 6 shows the additive effect of spongosine and gabapentin in the chronic constriction injury model of neuropathic pain;

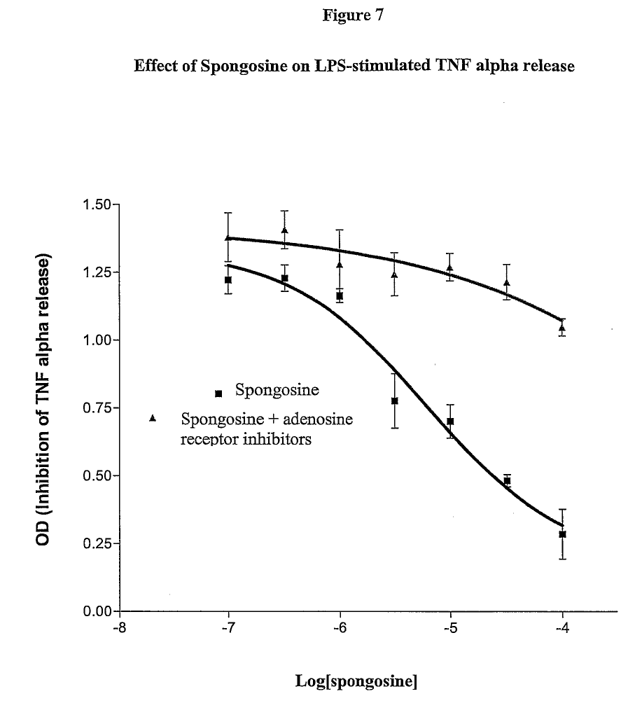

FIG. 7 shows the effect of spongosine on LPS induced TNF alpha release in cells of human macrophage cell line U937; and

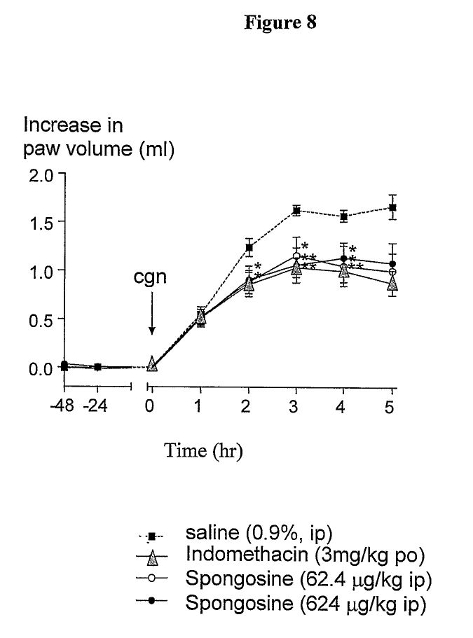

FIG. 8 shows that spongosine (62.4 and 624 μg/kg i.p.) inhibits carrageenan (CGN) induced inflammation with comparable efficacy to indomethacin (3 mg/kg, po), at concentrations that do not affect blood pressure.

Structures of preferred compounds of the invention are given in the Examples below. A Ki value is given for each compound at pH 5.5 and pH 7.4. To calculate this, rat striatal membranes were incubated for 90 minutes at 22° C. in the presence of 2 nM [3H]-CGS21680, 1 Unit/ml adenosine deaminase and increasing concentrations of the compound being studied, prior to filtration and liquid scintillation counting.

### EXAMPLE 1

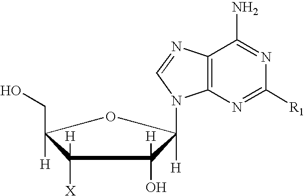

When X = OH

| Compound | Structure | (Ki) nM | (Ki) nM |
|---|---|---|---|
| No. | R₁ | (pH 5.5) | (pH 7.4) |
| 1 | OCH₃ | 1.5 | 1300 |
| 2 | OCH₂CHF₂ | 17 | 780 |
| 3 | OCH₂Cyclopropyl | 39 | 670 |
| 4 | OCH₂CH₂CH₂CH₃ | 11 | 280 |
| 5 | O CH₂CH₂CH₂CH₂CH₂CH₃ | 3 | 1500 |
| 6 | OPh | 71 | 2500 |
| 7 | O-(4-cyano)Ph | 4 | 1300 |
| 8 | O-(3-Ph)Ph | 0.7 | 620 |
| 9 | O-(2,5-F₂)Ph | 16 | 2500 |
| 10 | O-(2,4-F₂)Ph | 16 | 6400 |
| 11 | O-(3,4-F₂)Ph | 63 | 3300 |
| 12 | O-(2,3,5-F₃)Ph | 46 | 5900 |
| 13 | O-(3-Me, 4-F)Ph | 43 | 3100 |
| 14 | O-(2-Me)Ph | 24 | 22000 |
| 15 | O-(3-Br)Ph | 35 | 590 |
| 16 | O-(4-Me)Ph | 3.4 | 720 |
| 17 | 5-indanyloxy | 12 | 760 |
| 18 | O-(3-CH(CH₃)₂)Ph | 16 | 560 |
| 19 | NHCH₃ | 24 | 1356 |
| 20 | NHCH₂CH₃ | 130 | 1200 |
| 21 | N(CH₃)₂ | 24 | 13350 |
| 22 | NH-(R)-sec-Butyl | 33 | 510 |
| 23 | NH-(S)-sec-Butyl | 29 | 1400 |
| 24 | NHCH₂CH₂CH₂CH₂CH₂CH₃ | 0.7 | 290 |
| 25 | NH-exo-norbornane | 5.5 | 120 |
| 26 | NHPh | 5 | 160 |
| 27 | NH-(4-MeO)Ph | 3 | 55 |
| 28 | NH-(4-F)Ph | 10 | 200 |
| 29 | NH-cyclopentyl | 2.0 | 420 |
| 30 | NH-cyclohexyl | 0.4 | 1000 |
| 31 | N—CH₃, N-CH₂CH₂CH(CH₃)₂ | 26 | 4000 |
| 32 | OCH₂cyclopentyl | 0.2 | 200 |
| 33 | SO₂CH₂CH₃ | 100 | 39000 |
| 34 | OCH₂CH₂OH | 4 | 203 |
| 35 | O-(2,2,3,3-tetrafluoro- | 11 | 220 |
| | cycloButyl) | | |
| 36 | CH₂CH₂CH₂CH₂CH₂CH₂CH₃ | 15 | 800 |
| 37 | 3,5-Me₂-Phenyl | 24 | 5500 |
| 38 | CN | 25 | 175 |
| 39 | CONH₂ | 23 | 610 |
| 40 | 0 CH2CH2CH2CH2CH2CH3 | 13 | 2990 |

### EXAMPLE 2

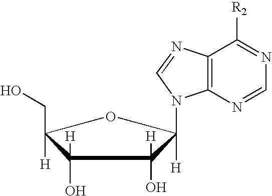

| Compound | Structure | (Ki) nM | (Ki) nM |
|---|---|---|---|
| No. | R₂ | (pH 5.5) | (pH 7.4) |
| 41 | N(CH₃)₂ | 42 | 450000 |
| 42 | NHCH₂CHC(CH₃)₂ | 91.5 | 8600 |
| 43 | N—CH₃, N—CH₂Ph | 7 | 18500 |
| 44 | N—CH₃, N—CH₂Ph(3-Br) | 29 | 7500 |
| 45 | N—CH₃, N—CH₂Ph(3-CF₃) | 3.8 | 20000 |
| 46 | Piperazinyl | 38 | 5000 |
| 47 | N—Me, N—(CH₂CH₂OCH₃) | 13 | 13000 |
| 48 | OCH₂Cyclopentyl | 140 | 21000 |

### EXAMPLE 3

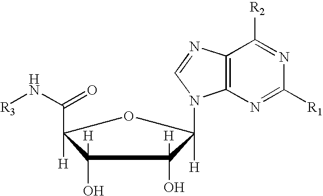

| Compound | | | | (Ki) nM | (Ki) nM |
|---|---|---|---|---|---|
| No. | R₁ | R₂ | R₃ | (pH 5.5) | (pH 7.4) |
| 49 | H | NH₂ | CH(CH₃)₂ | 5 | 1930 |
| 50 | H | NH₂ | H | 9 | 270 |
| 51 | H | NHCH₃ | CH(CH₃)₂ | 188 | 2440 |
| 52 | H | NH(CH₂)₂₂CHMe₂ | CH(CH₃)₂ | 39 | 1300 |
| 53 | OCH₃ | NH₂ | Ph | 230 | 26100 |
| 54 | NH(CH₂)₅Me | NH₂ | (CH₂)₃Me | 0.3 | 540 |

### EXAMPLE 4

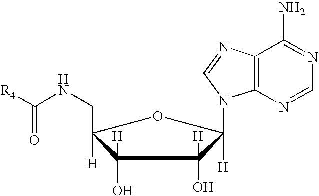

| Compound | Structure | (Ki) nM | (Ki) nM |
|---|---|---|---|
| No. | R₄ | (pH 5.5) | (pH 7.4) |
| 55 | CH₂CH₂CH₃ | 145 | 16900 |
| 56 | NHCH₂CH₃ | 40 | 6570 |

### EXAMPLE 5

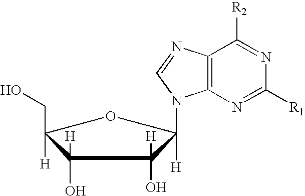

| Compound | | | (Ki) nM | (Ki) nM |
|---|---|---|---|---|
| No. | R₁ | R₂ | (pH 5.5) | (pH 7.4) |
| 57 | NHCyclohexyl | NMe₂ | 2.6 | 18000 |
| 58 | OMe | NHBenzyl | 4.5 | 6100 |

### EXAMPLE 6

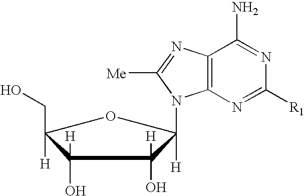

| Compound | | (Ki) nM | (Ki) nM |
|---|---|---|---|
| No. | R₁ | (pH 5.5) | (pH 7.4) |
| 59 | NHCyclohexy | 31 | 3900 |
| 60 | NHCyclopentyl | 49 | 2400 |
| 61 | NH-n-Hexyl | 130 | 4800 |

### EXAMPLE 7

FIG. 1: Spongosine (0.624 mg/kg p.o.) has no significant effect on blood pressure or heart rate. An implantable radiotelemetry device was placed in the abdominal cavity of 6 rats per group. The pressure catheter of the device was inserted in the abdominal aorta and two electrodes tunnelised under the skin in a lead II position (left side of abdominal cavity/right shoulder). Individual rats were placed in their own cage on a radioreceptor (DSI) for data acquisition. A: blood pressure; B: heart rate.

### EXAMPLE 8

The EC50 value of spongosine at adenosine receptors (measured at pH7.4) is 900 ng/ml (3 μM). FIG. 2 shows the change in plasma concentration over time after administration of spongosine at 0.6 mg/kg to a rat. It can be seen that the plasma concentration remains above 2% of the EC50 value for more than 3 hours. Anti-hyperalgesic effects have been observed (without blood pressure changes) when the peak plasma concentration is between 1% and 30% of the EC50 value determined in vitro. If the peak plasma concentration reaches the EC50 value profound reductions in blood pressure occur that last for hours.

### EXAMPLE 9

FIG. 3: A. Spongosine (0.624 mg/kg p.o.) inhibits carrageenan (CGN) induced thermal hyperalgesia (CITH) with comparable efficacy to indomethacin (3 mg/kg, po). B. Concentration-response relationship for Spongosine at 3 hrs post dosing. Carrageenan (2%, 10 microlitres) was administered into the right hind paw. A heat source was placed close to the treated and untreated hind paws, and the difference in the paw withdrawal latencies is shown. Spongosine was administered at the same time as carrageenan.

### EXAMPLE 10

FIG. 4: Spongosine (0.624 mg/kg p.o.) inhibits thermal hyperalgesia caused by chronic constriction injury of the rat sciatic nerve. Under anaesthesia the sciatic nerve was displayed in the right leg, and four loose ligatures tied round the nerve bundle. After approximately two weeks the rats developed thermal hyperalgesia in the operated leg as judged by the difference in paw withdrawal latencies of the right and left paws. Administration of spongosine reduced the hyperalgesia as shown by the reduction in the difference between the withdrawal latencies. Spongosine was as, or more, effective than carbamazepine (CBZ, 100 mg/kg s.c.)

### EXAMPLE 11

FIG. 5: Spongosine (1.2 mg/kg p.o.) inhibits static allodynia caused by chronic constriction injury of the rat sciatic nerve, both in the presence and absence of naloxone (1 mg/kg s.c.). Under anaesthesia the sciatic nerve was displayed in the right leg, and four loose ligatures tied round the nerve bundle. After approximately two weeks the rats developed static allodynia in the operated leg as judged by the difference in paw withdrawal thresholds of the right and left paws. Administration of spongosine reduced the hyperalgesia as shown by the increased paw withdrawal threshold (PWT) in the presence and absence of naloxone. Veh: vehicle.

### EXAMPLE 12

FIG. 6: Spongosine and gabapentin inhibit static allodynia caused by chronic constriction injury of the rat sciatic nerve. Spongosine and gabapentin were administered (p.o.) in different proportions as indicated in the drawing. The total dose administered is shown on the horizontal axis, and the paw withdrawal threshold (PWT) on the vertical axis. The predicted anti-hyperalgesic effect (derived from the dose response curves obtained with each agent alone) if the effects of the two compounds are additive is shown (). The observed effects are indicated by (▪). It is apparent that the observed effects are not significantly different from those predicted by additivity.

### EXAMPLE 13

Cells of human macrophage cell line U937 were grown in suspension to 500,000 cells/ml, plated out into 48 well plates, treated with 20 ng/ml PMA and incubated for 8 hours. Cells adhered to well bottoms and were washed and allowed to recover for 36 hours before use. Plates were preincubated with concentrations of spongosine, and 100 ng/ml of LPS was added 10 minutes later to stimulate TNF production. After 3 hours the cell supernatants were assayed for TNF alpha using fluorescence labeled ELISA kits. A graph showing the results (inhibition of TNF alpha release against spongosine concentration) is shown in FIG. 7. The results show that spongosine inhibits LPS induced TNF release, and that this inhibition is sensitive to adenosine receptor inhibitors.

### EXAMPLE 14

### Preparation of Compounds 2 and 32

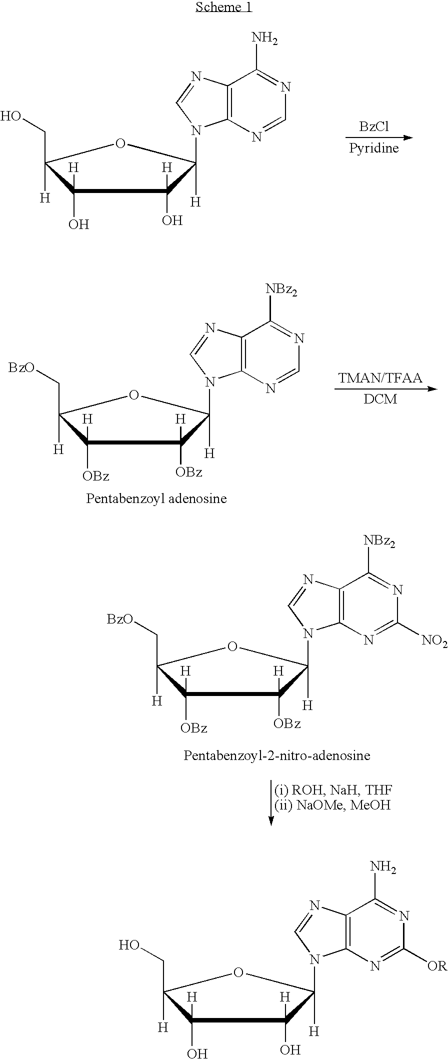

To a solution of adenosine (1 eq) in pyridine was added benzoyl chloride (7 eq) and the resulting solution was refluxed at 80° C. for 4 h. The solvents were removed in vacuo and the residue dissolved in EtOAc and washed with aq. NaHCO₃, brine and water, and the organic phase dried over MgSO₄. Crystallisation from dichloromethane (DCM)/EtOH afforded pentabenzoyl adenosine as a white solid.

To a solution of tetramethylammonium nitrate (TMAN) (1.5 eq) in DCM was added trifluoroacetic anhydride (TFAA) (1.5 eq) and the resulting solution stirred at rt for 1 h. The mixture was cooled to 0° C. and a solution of pentabenzoyl adenosine (1 eq) in DCM was added. The resulting solution was allowed to warm to rt over 4 h. The solution was then washed with aq. NaHCO₃, brine and water (×3) and the organic phase dried over MgSO₄. Crystallisation from DCM/EtOH afforded pentabenzoyl-2-nitro-adenosine as a pale yellow solid.

To a solution of alcohol ROH (R=CH₂CHF₂ (2) or CH₂cyclopentyl (32)) (1.5 eq) in THF was added NaH (1.5 eq) and the resulting suspension stirred for 1 h. The resulting solution was added to a solution of pentabenzoyl-2-nitro-adenosine (1 eq) in THF and stirring continued for 16 h. The solvents were then removed in vacuo and the residue dissolved in MeOH. NaOMe (4 eq) was then added and the resulting suspension stirred for 4 h before being quenched with aq. citric acid. The solvents were removed in vacuo and the residue purified by reverse phase column chromatography to yield the 2-alkoxy derivative.

### EXAMPLE 15

### Preparation of Compounds 3 and 35

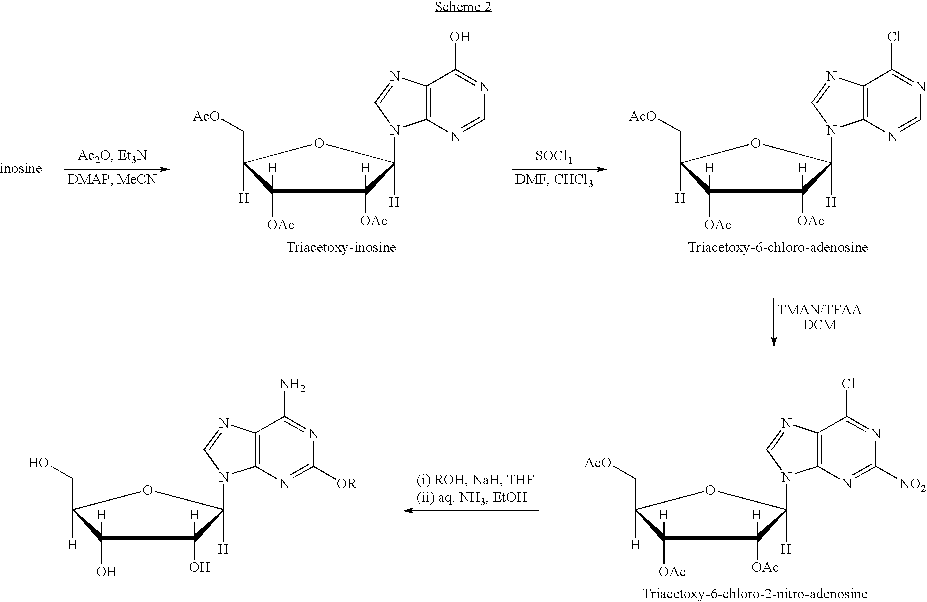

To a suspension of inosine (1 eq) and DMAP (0.1 eq) in MeCN was added Et₃N (3.8 eq) and acetic anhydride (3.5 eq) and the resulting mixture stirred for 1 h. MeOH was then added and stirring continued for 15 mins. The solvents were then removed in vacuo and the product triturated from isopropanol. The resulting solid was filtered and washed with isopropanol yielding triacetoxy inosine.

To a solution of triacetoxy inosine (1 eq) in CHCl₃ was added DMF (3 eq) and thionyl chloride (3 eq) and the resulting solution was refluxed for 16 h. The solvents were then removed in vacuo and the residue dissolved in DCM and washed with aq. NaHCO₃ and brine and the organic phase dried over MgSO₄ to afford triacetoxy-6-chloro-adenosine.

To a solution of tetramethylammonium nitrate (TMAN) (1.5 eq) in DCM was added trifluoroacetic anhydride (TFAA) (1.5 eq) and the resulting solution stirred at rt for 1 h. The mixture was cooled to 0° C. and a solution of triacetoxy-6-chloro-adenosine (1 eq) in DCM was added. The resulting solution was allowed to warm to rt over 2.5 h. The solution was then washed with aq. NaHCO₃, brine and water (×3) and the organic phase dried over MgSO₄. Crystallisation from DCM/EtOH afforded triacetoxy-6-chloro-2-nitro-adenosine as a pale yellow solid which was washed with water and EtOH.

To a solution of alcohol ROH (R=CH₂Cyclopropyl (3) or 2,2,3,3-tetrafluorocyclobutane (35)) (1.5 eq) in THF was added NaH (1.5 eq) and the resulting suspension stirred for 15 mins. The resulting solution was then added to a solution of triacetoxy-6-chloro-2-nitro-adenosine (1 eq) in THF and stirring continued for 2-6 h. The solvents were then removed in vacuo, EtOH and aq. NH₃ were added and the resulting solution was heated in a sealed tube at 80° C. for 16 h. The mixture was then cooled, the solvents were removed in vacuo and the residue purified by reverse phase column chromatography to yield the 2-alkoxy derivative.

### EXAMPLE 16

### Preparation of Compounds 7-18

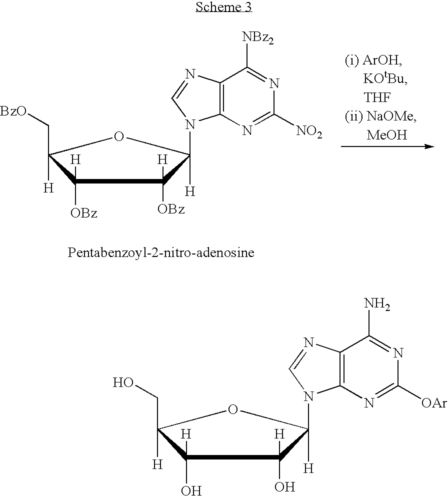

To a solution of phenol ArOH (Ar=4-cyanophenyl (7) or 3-phenyl-phenyl (8) or 2,5-difluorophenyl (9) or 2,4-difluorophenyl (10) or 3,4-difluorophenyl (11) or 2,3,5-trifluorophenyl (12) or 3-methyl,4-fluorophenyl (13) or 2-methylphenyl (14) or 3-bromophenyl (15) or 4-methylphenyl (16) or 5-indanyl (17) or 3-isopropylphenyl (18)) (1.5 eq) in THF was added KO^{t}Bu (1.5 eq) and the resulting suspension stirred for 30 min. The resulting solution was added to a solution of pentabenzoyl-2-nitro-adenosine (see scheme 1) (1 eq) in THF and stirring continued for 16 h. The solvents were then removed in vacuo and the residue dissolved in MeOH. NaOMe (4 eq) was then added and the resulting suspension stirred for 4 h before being quenched with aq. citric acid. The solvents were removed in vacuo and the residue purified by reverse phase column chromatography to yield the 2-aryloxy derivative.

### EXAMPLE 17

### Preparation of Compounds 22-25 and 31

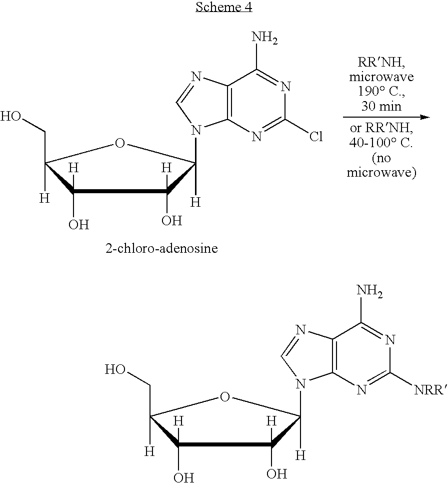

A solution of 2-chloroadenosine in neat amine RR′NH (RR′N=NH—(R)-sec-butyl (22) or NH—(S)-sec-butyl (23) or NH-n-Hexyl (24) or NH-exo-norbornane (25) or N(Me)isoamyl (31)) was either heated at 190° C. in a microwave for 30 min or heated at 40-100° C. for 16 h. The solvents were then removed in vacuo and the residue purified by reverse phase column chromatography to yield the 2-aminoalkyl derivative.

### EXAMPLE 18

### Preparation of Compound 33

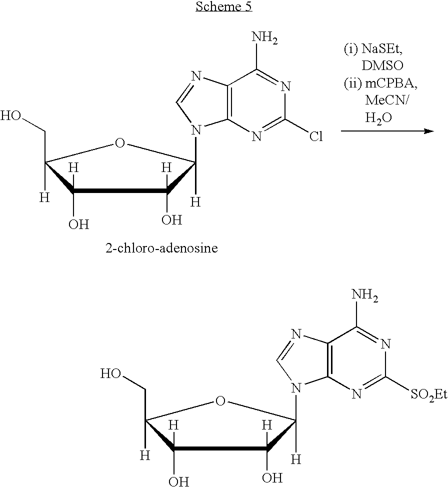

To a solution of 2-chloro-adenosine (1 eq) in DMSO was added NaSEt (1.3 eq) and the resulting solution heated at 80° C. for 20 h. The solvents were then removed in vacuo and the residue purified by reverse phase column chromatography to yield 2-ethylthio-adenosine.

To a solution of 2-ethylthio-adenosine in MeCN/H₂O (1:1) was added meta-chloro-perbenzoic acid (mCPBA) (3 eq) and stirring continued for 16 h. The solvents were then removed in vacuo and the residue purified by reverse phase column chromatography to yield 33.

### EXAMPLE 19

### Preparation of Compound 37

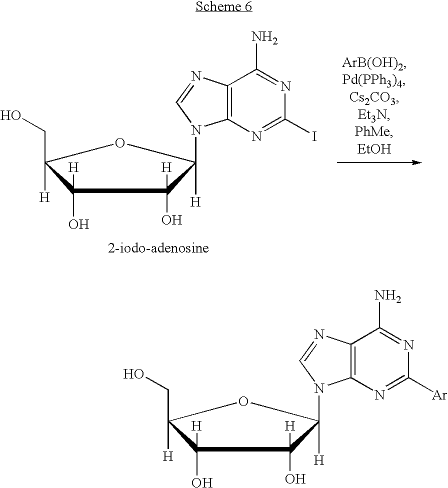

A suspension of 2-iodo-adenosine (1 eq), ArB(OH)₂ (Ar=3,5-dimethylphenyl) (1 eq), Pd(PPh₃)₄ (0.1 eq), Cs₂CO₃ (2.2 eq) and Et₃N (2.2 eq) in PhMe/EtOH (2:1) was heated at 110° C. for 16 h. The suspension was then filtered and the filtrate concentrated in vacuo and purified by reverse phase column chromatography to yield 37.

### EXAMPLE 20

### Preparation of Compound 40

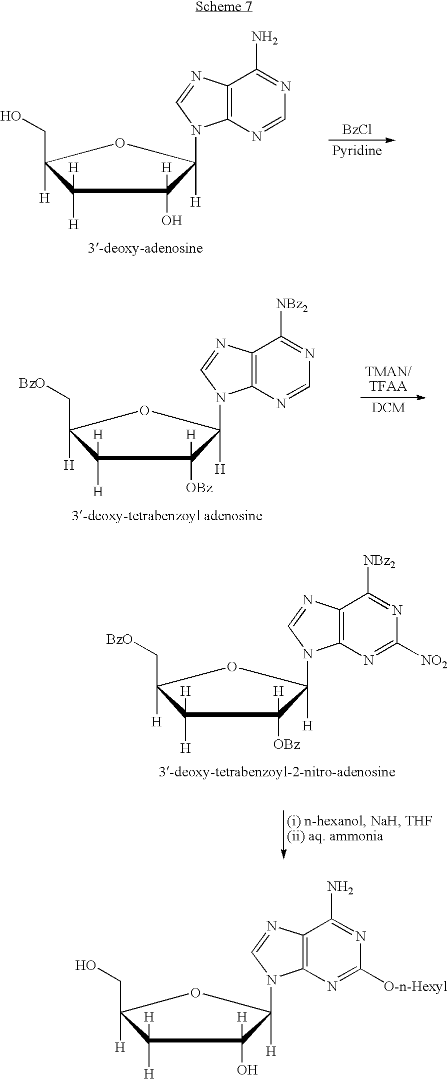

To a solution of 3′-deoxy-adenosine (1 eq) in pyridine was added benzoyl chloride (6 eq) and the resulting solution was refluxed at 65° C. for 4 h. The solvents were removed in vacuo and the residue dissolved in EtOAc and washed with water (×3) and brine, and the organic phase dried over MgSO₄. Purification using silica gel column chromatography afforded 3′-deoxy-tetrabenzoyl adenosine.

To a solution of TMAN (1.5 eq) in DCM was added TFAA (1.5 eq) and the resulting solution stirred at rt for 1 h. The mixture was cooled to 0° C. and a solution of 3′-deoxy-tetrabenzoyl adenosine (1 eq) in DCM was added. The resulting solution was allowed to warm to rt over 16 h. The solution was then washed with water (×3) and brine and the organic phase dried over MgSO₄ to give 3′-deoxy-tetrabenzoyl-2-nitro-adenosine.

To a solution of n-hexanol (2 eq) in THF was added NaH (2.1 eq) and the resulting suspension stirred for 30 min. The resulting solution was added to a solution of 3′-deoxy-tetrabenzoyl-2-nitro-adenosine (1 eq) in THF and stirring continued for 1 week. The solvents were then removed in vacuo and the residue purified by silica gel column chromatography. To a solution of this material in THF was added aq. NH₃ and the resulting suspension stirred for 12 h. The solvents were removed in vacuo and the residue purified by reverse phase column chromatography to yield 40.

### EXAMPLE 21

### Preparation of Compounds 44, 45 and 47

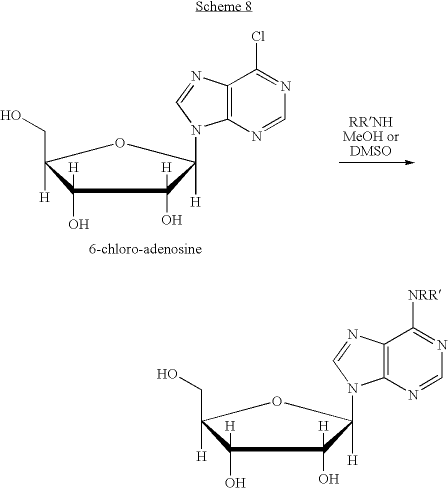

To a solution of 6-chloroadenosine (1 eq) in MeOH or DMSO was added amine RR′NH (RR′N=N(Me)CH₂(3-bromophenyl) (44) or N(Me)CH₂(3-trifluoromethylphenyl) (45) or N(Me)CH₂CH₂OMe (47)) (3-5 eq) and the resulting solution stirred for 16 h. The solvents were then removed in vacuo and the residue purified by reverse phase column chromatography to yield the 6-dialkylamino derivative.

### EXAMPLE 22

### Preparation of Compound 48

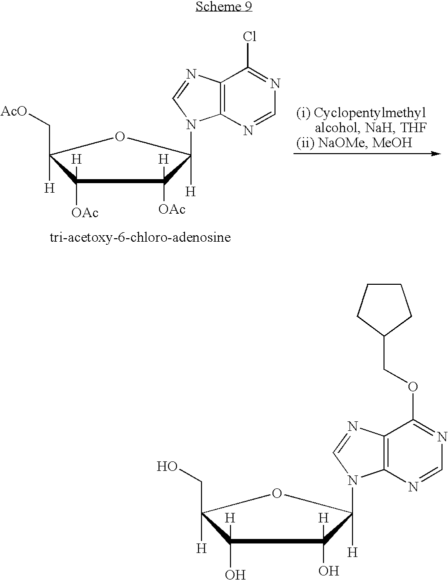

To a solution of cyclopentylmethyl alcohol (1.5 eq) in THF was added NaH (1.5 eq) and the resulting suspension stirred for 1 h. The resulting solution was added to a solution of triacetoxy-6-chloro-adenosine (see scheme 2) (1 eq) in THF and stirring continued for 16 h. The solvents were then removed in vacuo and the residue dissolved in MeOH. NaOMe (4 eq) was then added and the resulting suspension stirred for 4 h before being quenched with aq. citric acid. The solvents were removed in vacuo and the residue purified by reverse phase column chromatography to yield the 6-alkoxy derivative.

### EXAMPLE 23

### Preparation of Compounds 51 and 52

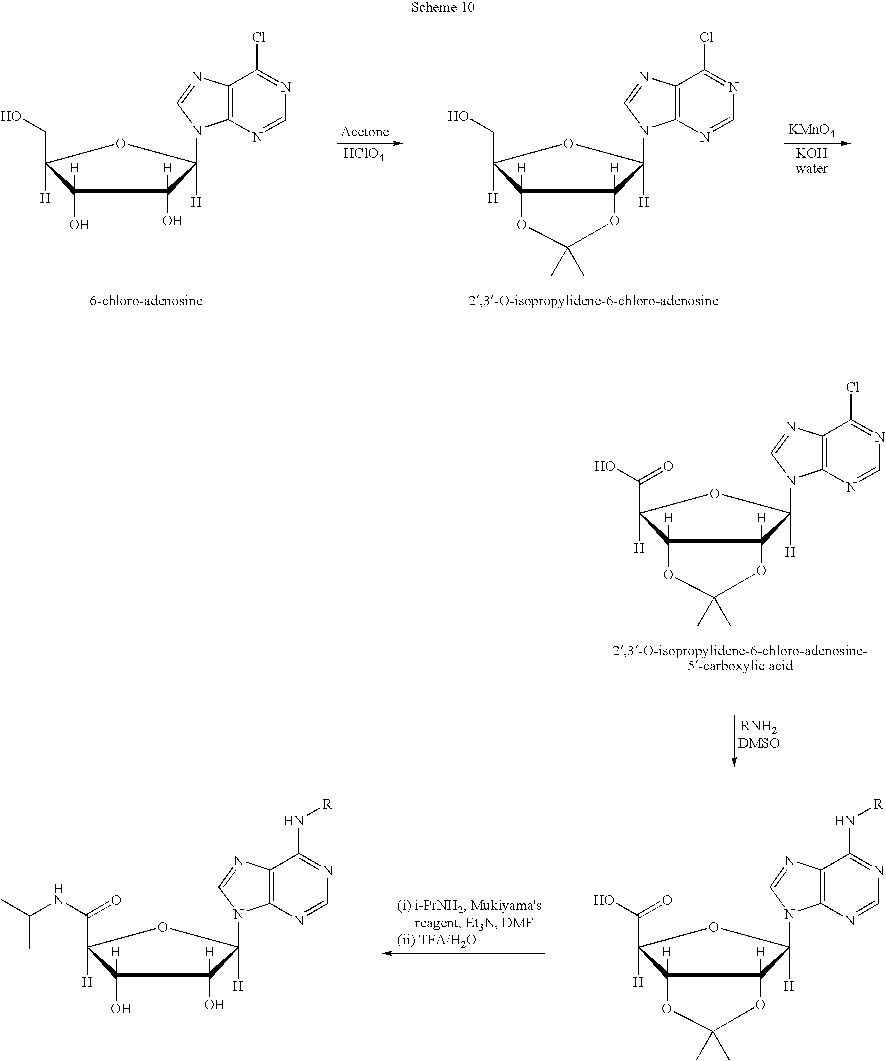

To a suspension of 6-chloro-adenosine in acetone at 0° C. was added HClO₄ and stirring continued for 2 h. Aq. NH₃ was then added and the solution concentrated in vacuo. The solution was cooled to −20° C. and the resulting white precipitate of 2′3′-O-isopropylidene-6-chloro-adenosine was collected and washed with acetone.

KOH (2.5 eq) and KMnO₄ (2.5 eq) were added to a suspension of 2′3′-O-isopropylidene-6-chloro-adenosine (1 eq) in water and stirring continued for 4 h. The reaction mixture was then quenched with hydrogen peroxide, concentrated and cooled to −20° C. The resulting precipitate was collected and washed with water to afford 2′3′-O-isopropylidene-6-chloro-adenosine-5′-carboxylic acid.

To a solution of 2′3′-O-isopropylidene-6-chloro-adenosine-5′-carboxylic acid (1 eq) in DMSO was added RNH₂ (R=Me (51) or isoamyl (52)) (2 eq) and the resulting solution stirred for 16 h. The solvents were then removed in vacuo and the residue purified by reverse phase column chromatography to afford the corresponding 2′3′-O-isopropylidene-6-alkylamino-adenosine-5′-carboxylic acid.

A solution of 2′3′-O-isopropylidene-6-alkylamino-adenosine-5′-carboxylic acid (1 eq), Muliyama's reagent (1.2 eq), isopropylamine (1.5 eq) and Et₃N (2.5 eq) in DMF was stirred for 6 h. The solvents were then removed in vacuo and the residue purified by reverse phase column chromatography. Treatment with trifluoroacetic acid (TFA)/water (2:1) for 2 h followed by removal of the solvents in vacuo and purification by reverse phase column chromatography afforded the title products.

### EXAMPLE 24

### Preparation of Compound 53

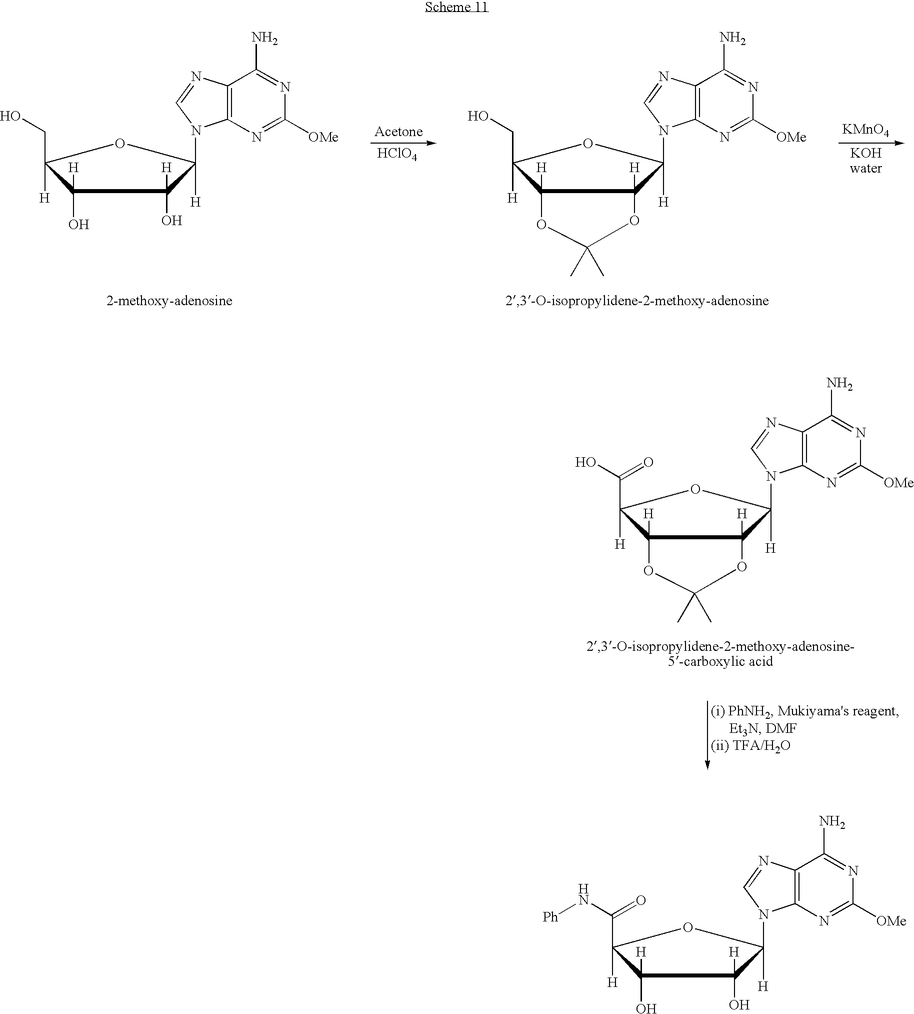

To a suspension of 2-methoxy-adenosine in acetone at 0° C. was added HClO₄ and stirring continued for 2 h. Aq. NH₃ was then added and the solvents were removed in vacuo and the residue purified by reverse phase column chromatography to yield 2′3′-O-isopropylidene-2-methoxy-adenosine.

KOH (2.5 eq) and KMnO₄ (2.5 eq) were added to a suspension of 2′3′-O-isopropylidene-2-methoxy-adenosine (1 eq) in water and stirring continued for 2 h. Further KOH (0.2 eq) and KMnO₄ (0.2 eq) were added and stirring continued for 4 h. The reaction mixture was then quenched with hydrogen peroxide, concentrated and cooled to −20° C. The resulting precipitate was collected and washed with water to afford 2′3′-O-isopropylidene-2-methoxy-adenosine-5′-carboxylic acid.

A solution of 2′3′-O-isopropylidene-2-methoxy-adenosine-5′-carboxylic acid (1 eq), Muidyama's reagent (1.2 eq), aniline (1.5 eq) and Et₃N (2.5 eq) in DMF was stirred for 6 h. DMSO/water (1:1) was then added and the resulting white solid filtered. This solid was dissolved in TFA/water (2:1) and stirring continued for 5 h. The solvents were then removed in vacuo and the residue purified by reverse phase column chromatography to yield 53.

### EXAMPLE 25

### Preparation of Compound 54

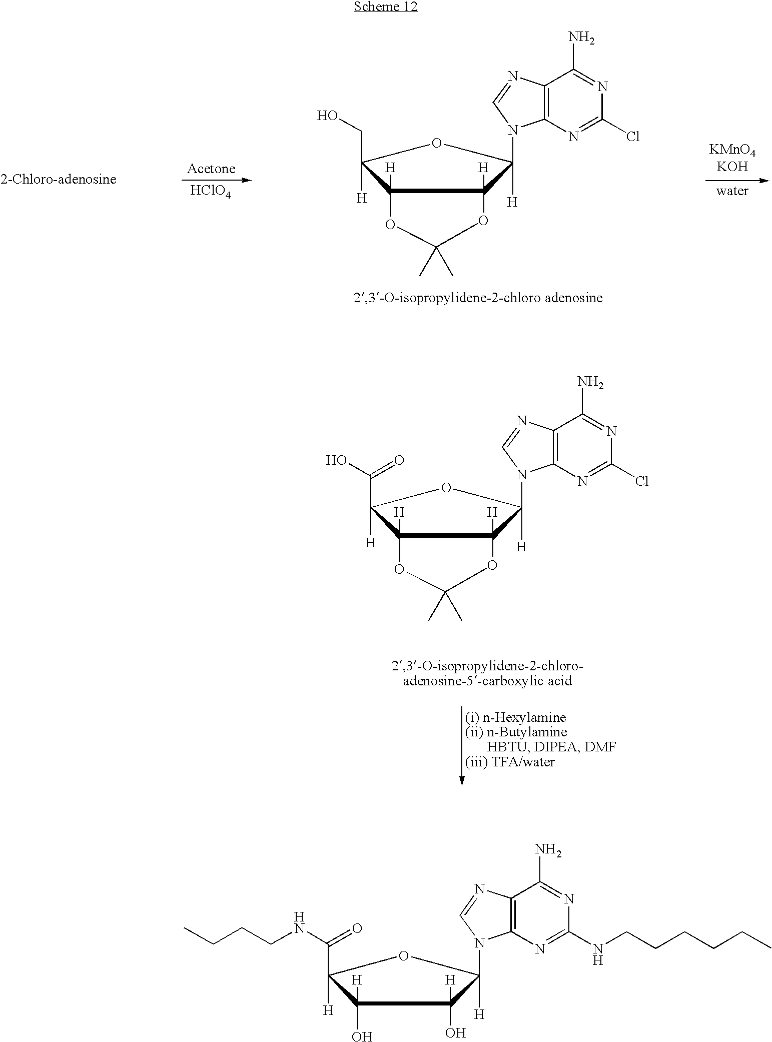

To a suspension of 2-chloro-adenosine in acetone at 0° C. was added HClO₄ and stirring continued for 2 h. Aq. NH₃ was then added and the solution concentrated in vacuo. The solution was cooled to −20° C. and the resulting white precipitate of 2′3′-O-isopropylidene-2-chloro-adenosine collected and washed with acetone.

KOH (2.5 eq) and KMnO₄ (2.5 eq) were added to a suspension of 2′3′-O-isopropylidene-2-chloro-adenosine (1 eq) in water and stirring continued for 4 h. The reaction mixture was then quenched with hydrogen peroxide, concentrated and cooled to −20° C. The resulting precipitate was collected and washed with water to afford 2′3′-O-isopropylidene-2-chloro-adenosine-5′-carboxylic acid.

A solution of 2′3′-O-isopropylidene-2-chloro-adenosine-5′-carboxylic acid (1 eq) in neat n-hexylamine was heated at 100° C. in a sealed tube for 24 h. Removal of the solvents in vacuo and purification by reverse phase column chromatography gave a pale brown solid (1 eq) which was dissolved in DMF at 0° C. To this solution was added n-Butylamine (4 eq), DIPEA (2.1 eq) and HBTU (1 eq) and stirring then continued at 0° C. for 4 h. The solvents were then removed in vacuo and the residue dissolved in EtOAc and washed with 0.2N HCl, aq. NaHCO₃ and brine and dried over MgSO₄ to give a yellow oil.

This oil was dissolved in TFA/water (2:1) and stirring continued for 2 h. The solvents were then removed in vacuo and the residue purified by reverse phase column chromatography to yield 54.

### EXAMPLE 26

### Preparation of Compound 55

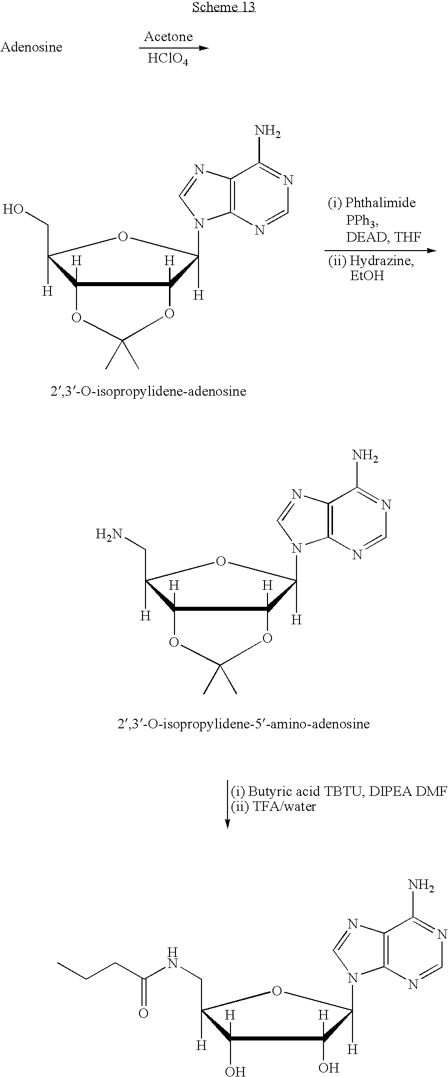

To a suspension of adenosine in acetone at 0° C. was added HClO₄ and stirring continued for 2 h. Aq. NH₃ was then added and the solution concentrated in vacuo. The solution was cooled to −20° C. and the resulting white precipitate of 2′3′-O-isopropylidene-adenosine collected and washed with acetone.

To a solution of 2′3′-O-isopropylidene-adenosine (1 eq) and triphenyl phosphine (1 eq) and phthalimide (1.03 eq) in THF under argon was added diethyl azodicarboxylate (1 eq) and the mixture was stirred for 10 h. The resulting precipitate was collected and washed with diethyl ether. To a solution of this solid (1 eq) in EtOH was added hydrazine (15 eq) and the solution was refluxed for 2 h and then cooled to rt. The resulting precipitate was filtered, dissolved in water and adjusted to pH 4. The precipitate was filtered and the filtrate was adjusted to pH 10, extracted into chloroform and dried over MgSO₄ to afford 2′3′-O-isopropylidene-5′-amino-adenosine.

To a solution of butyric acid (1 eq) in DMF at 0° C. was added DIPEA (1.2 eq) and TBTU (1 eq) and stirring continued for 5 min. 2′3′-O-isopropylidene-5′-amino-adenosine (1 eq) was then added as a solution in DMF and the resulting solution stirred for 3 h. The crude product was then extracted into DCM, washed with water (×3) and dried over MgSO₄.

Treatment with TFA/water (2:1) for 2 h followed by removal of the solvents in vacuo and purification by reverse phase column chromatography afforded 55.

### EXAMPLE 27

### Preparation of Compound 56

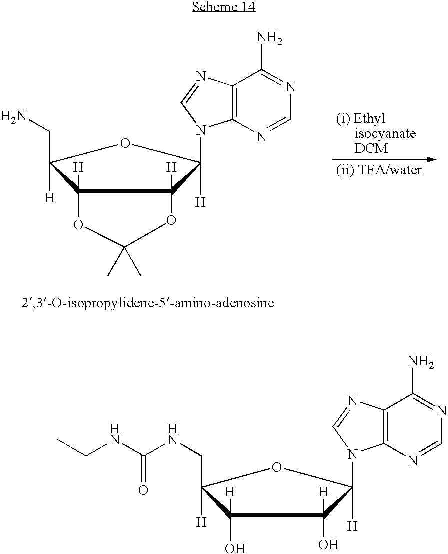

To a solution of 2′,3′-O-isopropylidene-5′-amino-adenosine (see scheme 13) (1 eq) in DCM was added ethyl isocyanate (1.2 eq) and stirring continued for 16 h. Polyamine resin was then added and filtered and the filtrate was concentrated in vacuo and purified by reverse phase column chromatography.

The resulting solid was dissolved in TFA/water (2:1) and stirring continued for 3 h. The solvents were then removed in vacuo and the residue purified by reverse phase column chromatography to yield 56.

### EXAMPLE 28

### Preparation of Compound 57

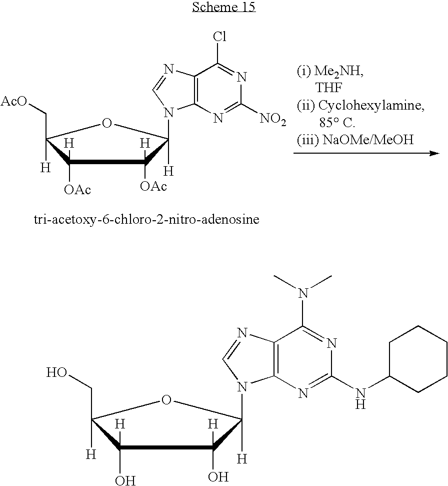

To a solution of triacetoxy-6-chloro-2-nitro-adenosine (see scheme 2) (1 eq) in THF was added dimethylamine (2 eq) and stirring continued for 4 h. Cyclohexylamine (1 eq) was then added and the resulting solution heated at 85° C. for 2 days. The solvents were then removed in vacuo and the residue dissolved in MeOH. NaOMe (1 eq) was then added and the resulting suspension stirred for 16 h. The solvents were removed in vacuo and the residue purified by reverse phase column chromatography to yield 57.

### EXAMPLE 29

### Preparation of Compound 58

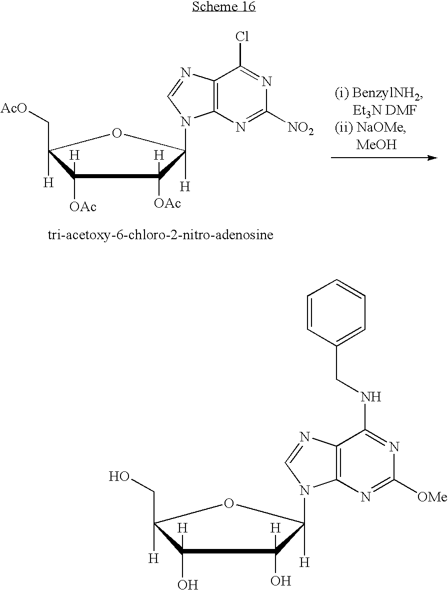

To a solution of triacetoxy-6-chloro-2-nitro-adenosine (see scheme 2) (1 eq) in DMF was added benzylamine (1 eq) and Et₃N (1.5 eq) and stirring continued for 10 mins. The solvents were then removed in vacuo and the residue purified by reverse phase column chromatography and dissolved in MeOH. NaOMe (2 eq) was then added and the resulting solution stirred for 4 h. The solvents were removed in vacuo and the residue purified by reverse phase column chromatography to yield 58.

### EXAMPLE 30

### Preparation of Compounds 59-61

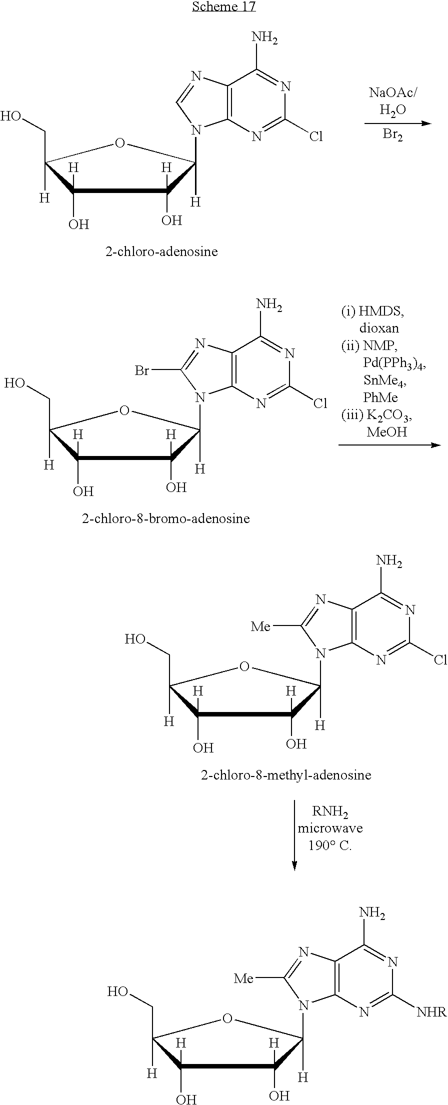

To a solution of 2-chloroadenosine (1 eq) in 1M aq. NaOAc (buffered to pH 4) was added bromine (1.2 eq) and the resulting solution stirred at rt for 16 h. The reaction mixture was then quenched with aq. NaHSO₃, adjusted to pH 7 and cooled to 4° C. The resulting precipitate of 2-chloro-8-bromo-adenosine was collected, washed with water and dried.

A solution of 2-chloro-8-bromo-adenosine (1 eq) in HMDS and dioxan was refluxed at 110° C. for 8 h. The solvents were then removed in vacuo, toluene added and the solvents again removed in vacuo. The residue was dissolved in N-Methylpyrrolidinone (NMP) and Pd(PPh₃)₄ (0.04 eq) and SnMe₄ (2 eq) were added and the resulting suspension was heated at 110° C. for 16 h. The solution was then cooled and the solvents removed in vacuo. The residue was dissolved in EtOAc and washed with water and the organic phase dried over MgSO₄. To a solution of the resulting oil in MeOH was added K₂CO₃ and the resulting suspension was stirred for 4 h. The solvents were then removed in vacuo and the residue purified by reverse phase column chromatography to yield 2-chloro-8-methyl adenosine.

A solution of 2-chloro-8-methyladenosine in neat amine RNH₂ (R=Cyclohexyl (59) or Cyclopentyl (60) or n-hexyl (61)) was heated at 190° C. in a microwave for 30 min. The solvents were then removed in vacuo and the residue purified by reverse phase column chromatography to yield the 8-methyl-2-aminoalkyl derivative.

### EXAMPLE 31

Plasma concentrations of spongosine were determined after single oral dosing in 5 or 6 human volunteers. Tachycardia was determined using 12 lead ECGs. The minimum effective analgesic plasma concentration in the rat was 0.025 μM suggesting a minimum effective dose in the human would be approximately 0.8 mg which results in plasma concentrations greater than 0.025 μM for approximately 1.5 h.

| | | Tachycardia |
|---|---|---|
| Dose | Plasma Cmax (μM) | side effect |
| 0.2 mg | 0.01 ± 0.005 | No |
| 0.8 mg | 0.04 ± 0.02 | No |
| 3.5 mg | 0.13 ± 0.04 | No |
| 10.5 mg | 0.3 ± 0.04 | No |
| 21 mg | 0.5 ± 0.1 | No |
| 28 mg | 0.6 ± 0.1 | Yes |

### EXAMPLE 32

Spongosine (62.4 and 624 μg/kg i.p.) inhibits carrageenan (CGN) induced inflammation with comparable efficacy to indomethacin (3 mg/kg, po), at concentrations that do not affect blood pressure. Carrageenan (2%, 10 microlitres) was administered into the right hind paw of a rat, and the paw volume assessed by plethysomometry. Spongosine was administered at the same time as carrageenan. The results are shown in FIG. 8. Spongosine was as effective as indomethacin (Indo, 3 mg/kg p.o.).

1. A compound of the following general formula, or a pharmaceutically acceptable salt thereof, for use as a medicament: 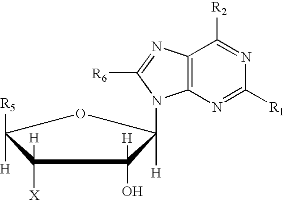
wherein:
(I) when X=OH, R₂=NH₂, R₅=CH₂OH, R₆=H, R₁ is C₅-C₆ alkoxy, OCH₂Cyclopropyl, OCH₂Cyclopentyl, O-(2,2,3,3-tetrafluoro-cycloButyl), phenoxy, substituted phenoxy, OCH₂CH₂OH, or OCH₂CHF₂, (5-indanyl)oxy, C₁, C₂, C₅, or C₆ alkylamino, (R) or (S)-sec-Butylamino, C₅ or C₆ cycloalkylamino, exo-norbornane amino, (N-methyl, N-isoamylamino), phenylamino, phenylamino with either methoxy or fluoro substituents, a C₂ sulfone group, a C₇ alkyl group, a cyano group, a CONH₂ group, or 3,5-dimethylphenyl; or
when X=H, R₂=NH₂, R₅=CH₂OH, R₆=H, R₁ is n-hexyloxy; or
(II) when X=OH, R₁=H, R₅=CH₂OH, R₆=H, R₂ is NMe₂, N-(2-isopentenyl), piperazinyl, (N-Me, N-benzyl), (N-Me, N—CH₂Ph(3-Br)), (N-Me, N—CH₂Ph(3-CF₃)), or (N-Me, N-(2-methoxyethyl)), or OCH₂Cyclopentyl; or
(III) when X=OH, R₅=CONHR₃, R₆=H:
R₁ is H, R₃ is an isopropyl group, and R₂ is either NH₂ or a methylamino group (NHMe) or an isoamyl group (CH₂CH₂CHMe₂); or
R₁ is H, R₃ is H, and R₂ is NH₂; or
R₁ is OMe, R₃ is Ph, and R₂ is NH₂; or
R₁ is NHCH₂CH₂CH₂CH₂CH₂Me, R₃ is CH₂CH₂CH₂Me, and R₂ is NH₂; or
(IV) when X=OH, R₁=H, R₂=NH₂, R₅=CH₂NHCOR₄, R₆=H, R₄ is n-propyl or NHCH₂CH₃; or
(V) when X=OH, R₅=CH₂OH, R₆=H:
R₁ is NHCyclohexyl when R₂ is NMe₂; or
R₁ is OMe when R₂ is NHBenzyl; or
(VI) when X=OH, R₂=NH₂, R₅=CH₂OH, R₆=Me, R₁ is NHCyclohexyl, NHCyclopentyl, or NH-n-Hexyl.

2. A compound according to formula (I) of claim 1, or a pharmaceutically acceptable salt thereof, for use as a medicament, wherein when X is OH, R₂ is NH₂, R₅ is CH₂OH, and R₆ is H, R₁ is phenoxy substituted with 4-nitrile, 4-methyl, 3-phenyl, 3-bromo, 3-isopropyl, 2-methyl, 2,4-difluoro, 2,5-difluoro, 3,4-difluoro, 2,3,5-trifluoro, or (3-methyl,4-fluoro).

3. A compound according to claim 1, with a structure as defined in any of Examples 1-6, or a pharmaceutically acceptable salt thereof, for use as a medicament.

4. A compound according to claim 3, with a structure corresponding to any of compound numbers 2, 3, 7-19, 22-25, 28, 31-33, or 35-60 as defined in Examples 1-6, or a pharmaceutically acceptable salt thereof, for use as a medicament.

5. A compound according to claim 3, with a structure corresponding to any of compound numbers 2, 3, 7-18, 22-25, 31-33, 35, 37, 40, 44, 45, 47, 48, or 51-60 as defined in Examples 1-6, or a pharmaceutically acceptable salt thereof, for use as a medicament.

6. Use of a pharmaceutically acceptable salt of a compound of formula (VII) in the manufacture of a medicament for the prevention, treatment, or amelioration of a pathological condition that can be improved or prevented by agonism of adenosine A2A receptors: 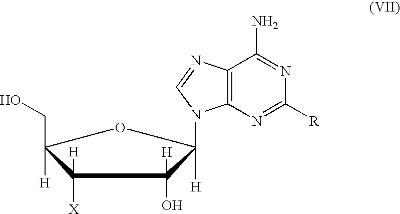
wherein: R is C₁₋₄ alkoxy, and X is H or OH.

7-28. (canceled)

29. A compound with a structure corresponding to any of compound numbers 2, 3, 7-18, 22-25, 31-33, 35, 37, 40, 44, 45, 47, 48, or 51-60 as defined in Examples 1-6, or a pharmaceutically acceptable salt thereof.

30. A method of preventing, treating, or ameliorating a pathological condition that can be prevented or improved by agonism of adenosine A2A receptors, which comprises administering a compound as defined in claim 1 to a subject in need of such prevention, treatment, or amelioration.

31. A method of preventing, treating, or ameliorating a pathological condition that can be prevented or improved by agonism of adenosine A2A receptors, excluding pain, cancer, inflammation, auto-immune disease, ischemia-reperfusion injury, epilepsy, sepsis, septic shock, neurodegeneration (including Alzheimer's Disease), muscle fatigue and muscle cramp, which comprises administering a compound of formula (VII) as defined in claim 6 to a subject in need of such prevention, treatment, or amelioration.

32. A method of preventing, treating, or ameliorating pain which comprises administering a compound as defined in claim 1 to a subject in need of such prevention, treatment, or amelioration.

33. A method of preventing, treating, or ameliorating ischaemic pain which comprises administering a compound as defined in claim 1 to a subject in need of such prevention, treatment, or amelioration.

34. A method according to claim 33 for the prevention, treatment, or amelioration of ischaemic pain associated with coronary artery disease, peripheral artery disease, left ventricular hypertrophy, essential hypertension, acute hypertensive emergency, cardiomyopathy, heart insufficiency, exercise tolerance, chronic heart failure, arrhythmia, cardiac dysrhythmia, syncopy, arteriosclerosis, mild chronic heart failure, angina pectoris, Prinzmetal's (variant) angina, stable angina, exercise induced angina, cardiac bypass reocclusion, intermittent claudication (arteriosclerosis oblitterens), arteritis, diastolic dysfunction, systolic dysfunction, atherosclerosis, post ischaemia/reperfusion injury, diabetes (Types I or II), thromboembolisms, haemorrhagic accidents, or neuropathic or inflammatory pain arising from hypoxia-induced nerve cell damage.

35. A method of prevention, treatment, or amelioration of inflammation, which comprises administering a compound as defined in claim 1 to a subject in need of such prevention, treatment, or amelioration.

36. A method according to claim 35 for the prevention, treatment, or amelioration of inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); autoimmune damage (including multiple sclerosis, Guillam Barre Syndrome, myasthenia gravis); obesity; cardiovascular conditions associated with poor tissue perfusion and inflammation (such as atheromas, atherosclerosis, stroke, ischaemia-reperfusion injury, claudication, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), muscle fatigue or muscle cramp (particularly athletes' cramp), arthritis (such as rheumatoid arthritis, osteoarthritis, rheumatoid spondylitis, gouty arthritis), fibrosis (for example of the lung, skin and liver), sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), bacterial meningitis, TNF-enhanced HIV replication, TNF inhibition of AZT and DDI activity, osteoporosis and other bone resorption diseases, osteoarthritis, rheumatoid arthritis, infertility from endometriosis, fever and myalgia due to infection, cachexia secondary to cancer, cachexia secondary to infection or malignancy, cachexia secondary to acquired immune deficiency syndrome (AIDS), AIDS related complex (ARC), keloid formation, scar tissue formation, adverse effects from amphotericin B treatment, adverse effects from interleukin-2 treatment, adverse effects from OKT3 treatment, or adverse effects from GM-CSF treatment, and other conditions mediated by excessive anti-inflammatory cell (including neutrophil, eosinophil, macrophage and T-cell) activity.

37. A method of preventing, treating, or ameliorating macro or micro vascular complications of type 1 and 2 diabetes, retinopathy, nephropathy, autonomic neuropathy, or blood vessel damage caused by ischaemia or atherosclerosis which comprises administering a compound as defined in claim 1 to a subject in need of such prevention, treatment, or amelioration.

38. A method of slowing the progression of arthropathy, which comprises administering a compound as defined in claim 1 as a disease-modifying antirheumatic drug (DMARD) to a subject in need thereof.

39. A method according to claim 38, for slowing the progression of rheumatoid arthritis.

40. A method according to claim 30, wherein the compound is administered at a dose that gives rise to a peak plasma concentration of the compound that is less than the EC50 value of the compound at adenosine receptors at pH 7.4.

41. A method according to claim 30, wherein the compound is administered to the subject in an amount that results in a peak plasma concentration of the compound in the subject that is one ten thousandth to one half of the lowest EC50 value of the compound at adenosine receptors.

42. A method according to claim 30, wherein the compound is administered to the subject in an amount that results in a plasma concentration of the compound in the subject being maintained for more than one hour at one ten thousandth to one half of the lowest EC50 value of the compound at adenosine receptors.

43. A method according to claim 30, wherein the compound is administered to the subject in an amount that results in a peak plasma concentration of the compound in the subject that is one ten thousandth to one half of the lowest Kd value of the compound at adenosine receptors.

44. A method according to claim 30, wherein the compound is administered to the subject in an amount that results in a plasma concentration of the compound in the subject being maintained for more than one hour at one ten thousandth to one half of the lowest Kd value of the compound at adenosine receptors.

45. A method according to claim 30, wherein the compound is administered to the subject in an amount that is one ten thousandth to one half of the minimum amount of the compound that gives rise to bradycardia, hypotension or tachycardia side effects in animals of the same species as the subject to which the compound is administered.

46. A method according to claim 30, wherein the compound is administered at a dose that is one thousandth to one half of the minimum dose of the compound that gives rise to bradycardia, hypotension or tachycardia side effects in animals of the same species as the subject to which the dose is to be administered.

47. A method according to claim 46, wherein the dose is one hundredth to one half of the minimum dose that gives rise to the side effects.

48. A method according to claim 30, wherein the compound is administered to the subject in an amount that results in a plasma concentration of the compound in the subject being maintained for more than one hour at one ten thousandth to one half of the minimum plasma concentration of the compound that gives rise to bradycardia, hypotension or tachycardia side effects in animals of the same species as the subject to which the compound is administered.

49. A method according to claim 30, wherein the compound is administered at a dose that results in a plasma concentration of the compound that is maintained for more than one hour between one hundredth and one half of the minimum dose of the compound that gives rise to bradycardia, hypotension or tachycardia side effects in animals of the same species as the subject to which the compound is to be administered.

50. A method according to claim 30, wherein the compound is administered at a dose of less than 0.4 mg/kg.

51. A method according to claim 30, wherein the compound is administered at a dosage of 0.001 to 0.4 mg/kg.

52. A method according to claim 30, wherein the compound is administered at a dose of at least 0.003 mg/kg.

53. A method according to claim 30, wherein the compound is administered at a dose of 0.01 to 0.1 mg/kg.

54. A method according to claim 30, wherein the compound is administered orally, parenterally, sublingually, transdermally, intrathecally, transmucosally, intravenously, intramuscularly, subcutaneously, topically, or by inhaling.

55. A method according to claim 30, wherein the compound is administered at a frequency of 2 or 3 times per day.

56. A method according to claim 30, wherein the subject is a human subject.

57. A method according to claim 38, wherein the compound is spongosine or a pharmaceutically acceptable salt thereof.

58. A pharmaceutical composition in unit dose form comprising up to 500 mg of a compound as defined in claim 1, excluding 2-phenylamino adenosine, and a physiologically acceptable carrier, excipient, or diluent.

59. A pharmaceutical composition in unit dose form comprising up to 500 mg of a compound as defined in claim 1 together with an NSAID or a DMARD, and a physiologically acceptable carrier, excipient, or diluent.

60. A method of producing compound number 2 or 32 as defined in Example 1, which comprises reacting pentabenzoyl-2-nitro-adenosine with ROH, and deprotecting the reaction product to produce compound number 2 or 32, wherein R=CH₂CHF₂ or CH₂cyclopentyl.

61. A method of producing compound number 3 or 35 as defined in Example 1, which comprises reacting triacetoxy-6-chloro-2-nitro-adenosine with ROH, and deprotecting the reaction product to produce compound number 3 or 35, wherein R=CH₂Cyclopropyl or 2,2,3,3-tetrafluorocyclobutane.

62. A method of producing any of compound numbers 7-18 as defined in Example 1, which comprises reacting pentabenzoyl-2-nitro-adenosine with ArOH, and deprotecting the reaction product to produce any of compound numbers 7-18, wherein Ar=4-cyanophenyl, 3-phenyl-phenyl, 2,5-difluorophenyl, 2,4-difluorophenyl, 3,4-difluorophenyl, 2,3,5-trifluorophenyl, 3-methyl,4-fluorophenyl, 2-methylphenyl, 3-bromophenyl, 4-methylphenyl, 5-indanyl, or 3-isopropylphenyl.

63. A method of producing any of compound numbers 22-25 or 31 as defined in Example 1, which comprises reacting 2-chloroadenosine with RR′NH to produce any of compound numbers 22-25 or 31, wherein RR′N=NH—(R)-sec-butyl, NH—(S)-sec-butyl, NH-n-Hexyl, NH-exo-norbornane, or N(Me)isoamyl.

64. A method of producing compound number 33 as defined in Example 1, which comprises reacting 2-chloro-adenosine with NaSEt to produce 2-ethylthio-adenosine, then producing compound number 33 from the 2-ethylthio-adenosine.

65. A method of producing compound number 37 as defined in Example 1, which comprises reacting 2-iodo-adenosine with ArB(OH)₂, wherein Ar=3,5-dimethylphenyl.

66. A method of producing compound 40 as defined in Example 1, which comprises reacting 3′-deoxy-tetrabenzoyl-2-nitro-adenosine with n-hexanol, and deprotecting the reaction product to produce compound number 40.

67. A method of producing compound number 44, 45, or 47 as defined in Example 2, which comprises reacting 6-chloro-adenosine with RR′NH, wherein RR′N=N(Me)CH₂(3-bromophenyl), N(Me)CH₂(3-trifluoromethylphenyl), or N(Me)CH₂CH₂OMe.

68. A method of producing compound number 48 as defined in Example 2, which comprises reacting tri-acetoxy-6-chloro-adenosine with cyclopentylmethyl alcohol and deprotecting the reaction product to produce compound number 48.

69. A method of producing compound number 51 or 52 as defined in Example 3, which comprises reacting 2′,3′-O-isopropylidene-6-alkylamino-adenosine-5′-carboxylic acid of the following formula: 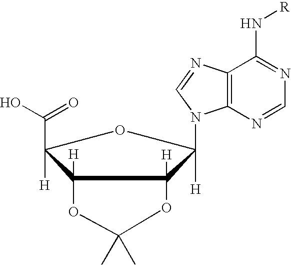
wherein R=Me or isoamyl;
with isopropylamine, and deprotecting the acetonide group of the reaction product to produce compound number 51 or 52.

70. A method of producing compound number 53 as defined in Example 3, which comprises reacting 2′,3′-O-isopropylidene-2-methoxy-adenosine-5′-carboxylic acid with aniline, and deprotecting the acetonide group of the reaction product to produce compound number 53.

71. A method of producing compound number 54 as defined in Example 3, which comprises reacting 2′,3′-O-isopropylidene-2-chloro-adenosine-5′-carboxylic acid with n-hexylamine, reacting the reaction product with n-Butylamine, and deprotecting the acetonide group of the product of the reaction with n-Butylamine to produce compound number 54.

72. A method of producing compound number 55 as defined in Example 4, which comprises reacting 2′,3′-O-isopropylidene-5′-amino-adenosine with butyric acid, and deprotecting the acetonide group of the reaction product to produce compound number 55.

73. A method of producing compound number 56 as defined in Example 4, which comprises reacting 2′,3′-O-isopropylidene-5′-amino-adenosine with ethyl isocyanate, and deprotecting the acetonide group of the reaction product to produce compound number 56.

74. A method of producing compound number 57 as defined in Example 5, which comprises reacting tri-acetoxy-6-chloro-2-nitro-adenosine with dimethylamine, reacting the reaction product with cyclohexylamine, and deprotecting the product of the reaction with cyclohexylamine to produce compound 57.

75. A method of producing compound number 58 as defined in Example 5, which comprises reacting tri-acetoxy-6-chloro-2-nitro-adenosine with benzylamine, and reacting the reaction product with methoxide anion and deprotecting the protected groups to produce compound 58.

76. A method of producing compound any of compounds 59-61 as defined in Example 6, which comprises reacting 2-chloro-8-methyl-adenosine with RNH₂, wherein R is Cyclohexyl, Cyclopentyl, or n-hexyl, to produce compound number 59, 60, or 61.
